(19) United States
(12) Patent Application Publication
(10) Pub. No.: US 2012/0321697 A1
(43) Pub. Date: Dec. 20, 2012

(54) BPB-BASED CARGO DELIVERY SYSTEM

(75) Inventors: Sang Yong Jon, Buk-gu (KR); Sung Hyun Kim, Buk-gu (KR); Seho Park, Buk-gu (KR); Dongkyu Kim, Buk-gu (KR); Jinho Park, Buk-gu (KR); Phei Er Saw, Buk-gu (KR)

(73) Assignee: GWANGJU INSTITUTE OF SCIENCE AND TECHNOLOGY, Buk-gu ,Gwangju (KR)

(21) Appl. No.: 13/515,163

(22) PCT Filed: Dec. 3, 2010
(86) PCT No.: PCT/KR2010/008645

§ 371 Date: Aug. 27, 2012

(30) Foreign Application Priority Data
Dec. 11, 2009 (KR) 10-2009-0123237

(51) Int. Cl. A61K 38/02; A61K 9/127; C07K 19/00; C07K 17/10; B82Y 5/00

(52) U.S. Cl. 424/450; 530/300; 530/322; 530/350; 514/1.1; 977/773

(57) ABSTRACT

Disclosed are bipodal peptide binder (BPB) based cargo delivery systems wherein cargo is linked to a BPB for delivery into cells or to cell surfaces using target binding ability and specificity of the BPB. The BPB has a structure stabilizing region of parallel or antiparallel amino acid strands or a combination of these strands to induce interstrand non-covalent bonds. The BPB also has target binding regions I and II, each binding to each of both termini of the structure stabilizing region. The number of amino acid residues of regions I and II, respectively, are n and m. The BPB exhibits the K_{D} value (dissociation constant) of a very low level (for example, nM level) and, therefore, exhibits very high affinity toward a biological target molecule. The BPB based cargo delivery system has applications in drugs, in vivo molecular imaging, in vitro cell imaging, drug delivery targeting and escort molecules.

### FIELD OF THE INVENTION

The present invention relates to a BPB-based cargo delivery system.

### DESCRIPTION OF THE RELATED ART

An antibody is an immunoglobulin protein as a serum protein which is produced by B cells, and specifically recognizes a particular region of foreign antigen to inactivate or incapacitate antigen. Using high-specification and high-affinity of antigen-antibody reaction and applying a variety of antibodies capable of discriminating 10 million antigens, numerous antibody products including diagnostics and therapeutics have been developed nowadays. Twenty one monoclonal antibodies have been approved by FDA until now, and antibodies such as Rituximab and Herceptin have been proved to have an excellent efficacy over 50% of subjects who exhibit no response to other therapies. In practice, the utilization of monoclonal antibodies results in successful clinic treatment including lymphoma, colorectal cancer or breast cancer. Whole market size of therapeutic antibodies might be evaluated to be in an annual average of 20% growth rate from 10 billion dollars in 2004 to 30 billion dollars in 2010 and predicted to be increased in a geometrical progression. There has been emerging focus on development of new drug using antibody because of: (a) short development period of drug; (b) economical investment cost; and (c) feasible prediction of adverse effects. Additionally, antibody as a herb medicine has no influence on a human body and is beneficial to a subject since it has half-life much longer than drugs with a low molecular weight. In spite of these availabilities, monoclonal antibodies may induce severe allergic or hypersensitive responses in human body due to recognition as a foreign antigen. Furthermore, clinical utilization of a monoclonal antibody with an anti-cancer activity has the following drawbacks: (a) high therapeutics cost due to high production cost; and (b) expensive licensing fees because intellectual property rights protect widespread techniques such as culture and purification method of antibodies.

To overcome these problems, it is earlier beginning to develop antibody alternatives in USA and EU. The antibody alternatives are designed as a recombinant protein having constant and variable domain like an antibody, of which the size is small and a particular region of a stable protein is replaced by random amino acid sequence, leading to produce a library, and the library is utilized for screening a target molecules to isolate a molecule with high affinity and excellent specificity. For example, it has been reported that avimer and affibody of antibody alternatives have a superior affinity to a target molecule in picomole level. Generally, the small-sized and stable antibody alternatives have been reported to penetrate into cancer cells in a feasible manner and to induce immune responses in a low level. First of all, the antibody alternatives may avoid antibody patent barriers and have excellent advantages such as (a) low production cost and (b) feasible massive purification from bacteria. Currently, 40 antibody alternatives have been known, and the example of antibody alternatives commercially attempted in ventures or international pharmaceuticals includes fibronectin type III domain, lipocalin, LDLR-A domain, crystalline, protein A, ankyrin repeat or BPTI protein, which have high affinity to a target molecule in the level of picomole. Of them, FDA clinic experiments for adnectin, avimer or Kunitz domain are on-going at present.

The present invention focused on a peptide-based antibody alternative different from conventionally protein-based antibody alternatives. Presently, peptides have been applied in a various manner to replace conventional antibody alternative therapeutics due to merits such as: (a) suitable pharmacokinetics; (b) massive production; (c) low cytotoxicity; (d) inhibition of antigenicity; and (e) low production cost. As a therapeutic drug, the advantage of peptide includes: (a) low production cost; (b) high safety and responsiveness; (c) relatively low patent royalty; (d) inhibition of antibody production against peptide in itself according to rare exposure on undesirable immune system; and (e) feasible modification and outstanding accuracy via synthesis. However, since most of peptides exhibits low affinity and specificity to a particular protein target compared with antibody, there is a drawback that they may be not utilized in several application fields. Therefore, it has been urgently demanded in the art to develop a novel peptide-based antibody alternative to overcome demerits of peptides. In this connection, the present inventors have made intensive studies to develop a peptide molecule capable of specifically binding a biological target molecule with high affinity. It should be expected as a technique capable of identifying a new drug with high affinity and specificity in a high-throughput manner using a peptide with low affinity reported about very numerous targets.

Throughout this application, various publications and patents are referred and citations are provided in parentheses. The disclosures of these publications and patents in their entities are hereby incorporated by references into this application in order to fully describe this invention and the state of the art to which this invention pertains.

### DETAILED DESCRIPTION OF THE INVENTION

### Technical Purposes of of the Invention

The present inventors have made intensive studies to develop a novel delivery system with a specific taget binding potential capable of deliverying various materials into cells or to cell surface. As results, we have developed a bipodal-peptide binder (BPB) with much more enhanced binding activity and specificity in which both termini of a structure stabilizing region having a relatively rigid peptide backbone are randomly linked to two peptides which are bound to a target molecule cooperatively. Furthermore, we have discovered that a cargo linked to the BPB can be delivered into cells or to cell surface with the help of target binding ability and specificity of the BPB.

Accordingly, it is an object of this invention to provide a BPB-based cargo delivery system.

Other objects and advantages of the present invention will become apparent from the following detailed description together with the appended claims and drawings.

### Technical Construction of the Invention

In one aspect of this invention, there is provided a BPB-based cargo delivery system, comprising:
(a) a bipodal peptide binder (BPB) comprising:
   (i) a structure stabilizing region comprising a parallel amino acid strand, an antiparallel amino acid strand or a parallel and an antiparallel amino acid strands to induce interstrand non-covalent bonds; and
   (ii) a target binding region I and a target binding region II each binding to each of both termini of the structure stabilizing region, wherein the number of amino acid residues of the target binding region I is n and the number of amino acid residues of the target binding region II is m; and
(b) a cargo linked to the bipodal peptide binder.

In another aspect of this invention, there is provided a method for deliverying a cargo, comprising: contacting to an individual, tissue or cell the BPB-based cargo delivery system comprising:
(a) a bipodal peptide binder (BPB) comprising: (i) a structure stabilizing region comprising a parallel amino acid strand, an antiparallel amino acid strand or a parallel and an antiparallel amino acid strands to induce interstrand non-covalent bonds; and (ii) a target binding region I and a target binding region II each binding to each of both termini of the structure stabilizing region, wherein the number of amino acid residues of the target binding region I is n and the number of amino acid residues of the target binding region II is m; and
(b) a cargo linked to the bipodal peptide binder.

The present inventors have made intensive studies to develop a novel delivery system with a specific taget binding potential capable of deliverying various materials into cells or to cell surface. As results, we have developed a bipodal-peptide binder (BPB) with much more enhanced binding activity and specificity in which both termini of a structure stabilizing region having a relatively rigid peptide backbone are randomly linked to two peptides which are bound to a target molecule cooperatively. Furthermore, we have discovered that a cargo linked to the BPB can be delivered into cells or to cell surface with the help of target binding ability and specificity of the BPB.

Basic strategy of this invention is to link peptides which are bound to both termini of a rigid peptide backbone. In this instance, the rigid peptide backbone functions to stabilize whole structure of a bipodal-peptide binder, and to reinforce that a target binding region I and a target binding region II are bound to a target molecule.

The structure stabilizing region capable of being utilized in the present invention includes a parallel amino acid strand, an antiparallel amino acid strand or a parallel and an antiparallel amino acid strands, and protein structure motifs in which non-covalent bonds are formed by an interstrand hydrogen bond, an electrostatic interaction, a hydrophobic interaction, a Van der Waals interaction, a pi-pi interaction, a cation-pi interaction or a combination thereof. Non-covalent bonds formed by an interstrand hydrogen bond, an electrostatic interaction, a hydrophobic interaction, a Van der Waals interaction, a pi-pi interaction, a cation-pi interaction or a combination thereof contributes to rigidity of a structure stabilizing region.

According to a preferable embodiment, the interstrand non-covalent bonds in the structure stabilizing region include a hydrogen bond, a hydrophobic interaction, a Van der Waals interaction, a pi-pi interaction or a combination thereof.

Alternatively, covalent bond may be involved in the structure stabilizing region. For example, disulfide bond in the structure stabilizing region permits to significantly enhance rigidity of the structure stabilizing region. Increase of rigidity caused by covalent bond is determined according to specificity and affinity of bipodal-peptide binder to a target.

According to a preferable embodiment, amino acid strands of the structure stabilizing region of the present invention are linked by a linker. The term “linker” used herein in the strand refers to a material which may link between strands. For instance, a turn sequence in a 3-hairpin used as a structure stabilizing region functions as a linker, and a material (e.g., peptide linker) linking between both C-termini in leucine zipper used as a structure stabilizing region functions as a linker.

Linker may link a parallel amino acid strand, an antiparallel amino acid strand or a parallel and an antiparallel amino acid strands. For example, at least two strands (preferably, two strands) arranged according to a parallel type, at least two strands (preferably, two strands) arranged according to an antiparallel type or at least three strands (preferably, three strands) arranged according to a parallel and an antiparallel type are linked by a linker.

According to a preferable embodiment, the linker of the present invention includes a turn sequence or a peptide linker.

According to a preferable embodiment, the turn sequence of the present invention includes a β-turn, a γ-turn, an α-turn, a π-turn or a ω-loop (Venkatachalam CM (1968), Biopolymers, 6, 1425-1436; Nemethy G and Printz M P. (1972), Macromolecules, 5, 755-758; Lewis P N et al., (1973), Biochim. Biophys. Acta, 303, 211-229; Toniolo C. (1980) CRC Crit. Rev. Biochem., 9, 1-44; Richardson J S. (1981), Adv. Protein Chem., 34, 167-339; Rose G D et al., (1985), Adv. Protein Chem., 37, 1-109; Milner-White E J and Poet R. (1987), TIBS, 12, 189-192; Wilmot C M and Thornton J M. (1988), J. Mol. Biol., 203, 221-232; Milner-White E J. (1990), J. Mol. Biol., 216, 385-397; Pavone V et al. (1996), Biopolymers, 38, 705-721; Rajashankar K R and Ramakumar S. (1996), Protein Sci., 5, 932-946). Most preferably, the turn sequence used in the present invention is a β-turn.

Example of β-turn used as a turn sequence includes preferably type I, type I′, type II, type II′, type III or type III′ turn sequence, more preferably type I, type I′, type II or type II′ turn sequence, much more preferably type I′ or type II′ turn sequence, and most preferably, type I′ turn sequence (B. L. Sibanda et al., J. Mol. Biol., 1989, 206, 4, 759-777; B. L. Sibanda et al., Methods Enzymol., 1991, 202, 59-82).

According to another preferable embodiment, the sequence capable of being used as a turn sequence in the present invention is disclosed in H. Jane Dyson et al., Eur. J. Biochem. 255:462-471(1998), which is incorporated herein by reference. The sequence capable of being used as a turn sequence in the present invention includes the following amino acid sequence: X-Pro-Gly-Glu-Val; or Alα-X-Gly-Glu-Val (X represents any amino acid selected from 20 amino acids).

According to one embodiment of this invention, it is preferable that two strands arranged according to a parallel type or two strands arranged according to an antiparallel type are linked by a peptide linker in β-sheet or leucine zipper used as a structure stabilizing region in the present invention.

It is possible in the present invention to utilize any peptide linker known to those ordinarily skilled in the art. The sequence of a suitable peptide linker may be selected by considering the following factor: (a) potential to be applied to a flexible extended conformation; (b) inability to form secondary structure capable of interacting with a biological target molecule; (c) absence of a hydrophobic or charged residue which interacts with a biological target molecule. Preferable peptide linkers include Gly, Asn and Ser residue. In addition, other neutral amino acid such as Thr and Ala may be included in a linker sequence. The amino acid sequence suitable in a linker is disclosed in Maratea et al., Gene 40:39-46(1985); Murphy et al., Proc. Natl. Acad Sci. USA 83:8258-8562(1986); U.S. Pat. Nos. 4,935,233, 4,751,180 and 5,990,275. Peptide linker sequence in the present invention may be composed of 1-50 amino acid residues.

According to a preferable embodiment, the structure stabilizing region of the present invention includes a β-hairpin motif, a β-sheet motif linked by a linker or a leucine-zipper motif linked by a linker, more preferably a β-hairpin motif or a β-sheet motif linked by a linker, and most preferably, a β-hairpin motif.

The term “β-hairpin” used herein means the most simple protein motif containing two β strands which are arranged each other in an antiparallel manner. Generally, two β strands in a β-hairpin are linked by a turn sequence.

Preferably, a turn sequence applied to a β-hairpin includes type I, type I, type II, type II, type III or type III′ turn sequence, more preferably type I, type I, type II or type II′ turn sequence, much more preferably type I′ or type II′ turn sequence, and most preferably, type I′ turn sequence. In addition, the following turn sequence may be utilized in a β-hairpin: X-Pro-Gly-Glu-Val; or Alα-X-Gly-Glu-Val (X represents any amino acid selected from 20 amino acids).

According to an illustrative example of the present invention, a type I turn sequence includes Asp-Asp-Alα-Thr-Lys-Thr, and a type I′ turn sequence includes Glu-Asn-Gly-Lys, and a type II turn sequence includes X-Pro-Gly-Glu-Val; or Alα-X-Gly-Glu-Val (X represents any amino acid selected from 20 amino acids), and a type II′ turn sequence includes Glu-Gly-Asn-Lys or Glu-D-Pro-Asn-Lys.

A peptide with β-hairpin conformation is well-known to those ordinarily skilled in the art, for example including tryptophan zipper motif disclosed in U.S. Pat. No. 6,914,123 and Andrea G. Cochran et al., PNAS, 98(10): 5578-5583), template-immobilized (β-hairpin mimetics in WO 2005/047503 and β-hairpin modifiers in U.S. Pat. No. 5,807,979. Besides, peptide with β-hairpin conformation is disclosed in Smith & Regan (1995) Science 270:980-982; Chou & Fassman (1978) Annu. Rev. Biochem. 47:251-276; Kim & Berg (1993) Nature 362:267-270; Minor & Kim (1994) Nature 367:660-663; Minor & Kim (1993) Nature 371:264-267; Smith et al. Biochemistry (1994) 33:5510-5517; Searle et al. (1995) Nat. Struct. Biol. 2:999-1006; Hague & Gellman (1997) J. Am. Chem. Soc. 119:2303-2304; Blanco et al. (1993) J. Am. Chem. Soc. 115:5887-5888; de Alba et al. (1996) Fold. Des. 1: 133-144; de Alba et al. (1997) Protein Sci. 6:2548-2560; Ramirez-Alvarado et al. (1996) Nat. Struct. Biol. 3:604-612; Stanger & Gellman (1998) J. Am. Chem. Soc. 120:4236-4237; Maynard & Searle (1997) Chem. Commun. 1297-1298; Griffiths-Jones et al. (1998) Chem. Commun. 789-790; Maynard et al. (1998) J. Am. Chem. Soc. 120:1996-2007; and Blanco et al. (1994) Nat. Struct. Biol. 1:584-590, which are incorporated herein by reference.

Most preferably, a peptide with β-hairpin conformation as a structure stabilizing region utilizes a tryptophan zipper motif.

According to a preferable embodiment, the tryptophan zipper used in the present invention is represented by the following Formula I:

X₁-Trp(X₂)X₃-X₄-X₅(X′₂)X₆-X₇ Formula I

wherein X₁ represents Ser or Gly-Glu, and X₂ and X′₂ independently represent Thr, His, Val, Ile, Phe or Tyr, and X₃ represents Trp or Tyr, and X₄ represents type I, type I′, type II, type II′, type III or type III′ turn sequence, and X₅ represents Trp or Phe, and X₆ represents Trp or Val, and X₇ represents Lys or Thr-Glu.

More preferably, X₁ represents Ser or Gly-Glu, and X₂ and X′₂ independently represent Thr, His or Val, and X₃ represents Trp or Tyr, and X₄ represents type I, type I′, type II or type II′ turn sequence, and X₅ represents Trp or Phe, and X₆ represents Trp or Val, and X₇ represents Lys or Thr-Glu in the Formula I.

Much more preferably, X₁ represents Ser or Gly-Glu, and X₂ and X′₂ independently represent Thr, His or Val, and X₃ represents Trp, and X₄ represents type I, type I′, type II or type II′ turn sequence, and X₅ represents Trp, and X₆ represents Trp, and X₇ represents Lys or Thr-Glu in the Formula I.

Still much more preferably, X₁ represents Ser, and X₂ and X′₂ represent Thr, and X₃ represents Trp, and X₄ represents type I′ or type II′ turn sequence, and X₅ represents Trp, and X₆ represents Trp, and X₇ represents Lys in the Formula I.

Most preferably, X₁ represents Ser, and X₂ and X′₂ represent Thr, and X₃ represents Trp, and X₄ represents type I′ turn sequence (ENGK) or type II′ turn sequence (EGNK), and X₅ represents Trp, and X₆ represents Trp, and X₇ represents Lys in the Formula I.

An illustrative amino acid sequence of tryptophan zipper suitable in the present invention is described in SEQ ID NOs: 1-3 and SEQ ID NOs: 5-10.

Another β-hairpin peptide capable of being utilized as a structure stabilizing region in the present invention includes a peptide derived from B1 domain of protein G, i.e. GB1 peptide.

Preferably, the GB1 peptide as a structure stabilizing region used in the present invention is represented by the following Formula II:

X₁-Trp-X₂-Tyr-X₃-Phe-Thr-Val-X₄ Formula II

wherein X₁ represents Arg, Gly-Glu or Lys-Lys, and X₂ represents Gln or Thr, and X₃ represents type I, type I′, type II, type II′, type III or type III′ turn sequence, and X₄ represents Gln, Thr-Glu or Gln-Glu.

More preferably, the structure stabilizing region in the Formula II is is represented by the following Formula II′:

X₁-Trp-Thr-Tyr-X₂-Phe-Thr-Val-X₃ Formula II′

wherein X₁ represents Gly-Glu or Lys-Lys, and X₂ represents type I, type I′, type

II, type II′, type III or type III′ turn sequence, and X₃ represents Thr-Glu or Gln-Glu.

An exemplified amino acid sequence of GB1 3-hairpin suitable in the present invention is described in SEQ ID NO: 4 and SEQ ID NOs: 14-15.

Betα-hairpin peptide capable of being utilized as a structure stabilizing region in the present invention includes a HP peptide.

Preferably, the HP peptide as a structure stabilizing region used in the present invention is represented by the following Formula III:

X₁-X₂-X₃-Trp-X₄-X₅-Thr-X₆-X₇ Formula III

wherein X₁ represents Lys or Lys-Lys, and X₂ represents Trp or Tyr, and X₃ represents Val or Thr, and X₄ represents type _{I,} type I′, type II, type II′, type III or type III′ turn sequence, and X₅ represents Trp or Ala, and X₆ represents Trp or Val, and X₇ represents Glu or Gln-Glu.

Still another β-hairpin peptide capable of being utilized as a structure stabilizing region in the present invention is represented by the following Formula IV:

X₁-X₂-X₃-Trp-X₄ Formula IV

wherein X₁ represents Lys-Thr or Gly, and X₂ represents Trp or Tyr, and X₃ represents type I, type I′, type II, type II′, type III or type III′ turn sequence, and X₄ represents Thr-Glu or Gly.

An illustrative amino acid sequence of β-hairpin in Formula III and IV is described in SEQ ID NOs: 11-12, SEQ ID NO: 15 and SEQ ID NOs: 16-19.

Accoridng to the present invention, a hairpin (e.g., alphα-hairpin, betα-hairpin gammα-hairpin and p-hairpin) as the structure stabilizing region may be used. In addition, β-turn as the structure stabilizing region may be used.

According to the present invention, a β-sheet linked by a linker may be used as a structure stabilizing region. The structure of β-sheet includes an extended form of two strands arranged in a parallel or antiparallel manner, preferably in an antiparallel manner, and hydrogen bond is formed between two strands.

Both adjacent termini of two amino acid strands in a β-sheet structure are linked by a linker. As described above, various turn-sequences or peptide linkers may be utilized as a linker. Using a turn sequence as a linker, it is most preferable to utilize a β-turn sequence.

According to another modified embodiment, a leucine zipper motif or a leucine zipper motif linked by a linker may be used as a structure stabilizing region. Leucine zipper motif is a conservative peptide domain which causes a dimerization of two parallel α-chains and a dimerization domain found generally in a protein related to gene expression (“Leucine scissors”. Glossary of Biochemistry and Molecular Biology (Revised). (1997). Ed. David M. Glick. London: Portland Press; Landschulz W H, et al. (1988) Science 240:1759-1764). In general, leucine zipper motif includes a haptad repeat sequence, and a leucine residue is located at fourth or fifth position. For example, a leucine zipper motif capable of being utilized in the present invention includes amino acid sequences such as LEALKEK, LKALEKE, LKKLVGE, LEDKVEE, LENEVAR and LLSKNYH. Practical example of leucine zipper motif used in the present invention is described in SEQ ID NO: 39. Half of each leucine zipper motif is composed of a short α-chain, and includes direct leucine interaction between α-chains. In general, leucine zipper motif in a transcription factor consists of a hydrophobic leucine zipper region and basic region (a region interacting with a major groove of DNA molecule). A basic region is not necessary for the leucine zipper motif used in the present invention. In the structure of leucine zipper motif, both adjacent termini of two amino acid strands (ie., two α-chains) may be linked by a linker. As described above, various turn-sequences or peptide linkers may be utilized as a linker. It is preferable to utilize a peptide linker which has no influence on the structure of leucine zipper motif.

Random amino acid sequence is linked in both termini of the above-mentioned structure stabilizing region. The random amino acid sequence forms a target binding region I and a target binding region II. It is one of the most features of the present invention that a peptide binder is constructed by a bipodal type which a target binding region I and a target binding region II are linked to both termini of a structure stabilizing region, respectively. The target binding region I and the target binding region II bind in a cooperative manner to a target, leading to enhance significantly affinity to the target.

The number (n) of amino acid residues of a target binding region I is not particularly limited, and is an integer of preferably 2-100, more preferably 2-50, much more preferably 2-20 and most preferably, β−10.

The number (m) of amino acid residues of a target binding region II is not particularly limited, and is an integer of preferably 2-100, more preferably 2-50, much more preferably 2-20 and most preferably, β−10.

The number of amino acid residuce of a target binding region I and a target binding region II may be independently different or equivalent. The amino acid sequence of a target binding region I and a target binding region II may be independently different or equivalent, and preferably independently different.

A sequence contained in a target binding region I and/or a target binding region II includes linear or circular amino acid sequence. To enhance stability of peptide sequence in the target binding regions, at least one amino acid residues of amino acid sequence contained in a target binding region I and/or a target binding region II may be modified into an acetyl group, a fluorenyl methoxy carbonyl group, a formyl group, a palmitoyl group, a myristyl group, a stearyl group or a polyethyleneglycol (PEG).

The bipodal-peptide binder of the present invention to be bound to a biological target molecule may be utilized in: regulation of in vivo physiological response;

detection of in vivo material; in vivo molecule imaging; in vitro cell imaging; targeting for drug delivery; and escort molecule.

According to a preferable embodiment, the cargo is linked to the structure stabilizing region, the target binding region I or the target binding region II (more preferably, the structure stabilizing region and much more preferably, the linker of the structure stabilizing region). The cargo includes, but not limited to, a chemical compound, a chemical drug, a biodrug, an inorganic particle, a nanoparticle, a protein, a peptide, a nucleic acid molecule, a lipid, a carbohydrate, a liposome or a label capable of generating a detectable signal.

The label capable of generating a detectable signal includes, but is not limited to, T1 contrast materials (e.g., Gd chelate compounds), T2 contrast materials [e.g., superparamagnetic materials (example: magnetite, Fe₃O₄, γ-Fe₂O₃, manganese ferrite, cobalt ferrite and nickel ferrite)], radioactive isotope (example: ¹¹C, ¹⁵O, ¹³N, P³², S³⁵, ⁴⁴Sc, ⁴⁵Ti, ¹¹⁸I, ¹³⁶La, ¹⁹⁸Ti, ²⁰⁵Bi and ²⁰⁶Bi), fluorescent materials (fluorescein, phycoerythrin, rhodamine, lissamine, and Cy3/Cy5), chemiluminescent materials, magnetic particles, mass labels and dense electron particle.

For example, the chemical drug includes an anti-flammatory agent, an analgesic, an anti-arthritic agent, an antispasmodic agent, an anti-depressant, an anti-psychotic agent, a sedative, an anti-anxiety drug, a drug antagonist, an anti-Parkinson's disease drug, a choline agonist, an anti-cancer drug, an anti-angiogenesis inhibitor, an immunosuppressive agent, an anti-viral agent, an antibiotics, an appetite depressant, an anti-choline agent, an anti-histamine agent, an anti-migraine medication, a hormone agent, a coronary, cerebrovascular or perivascular vasodilator, a contraceptive, an anti-thrombotic agent, a diuretic agent, an anti-hypertensive agent, a cardiovascular disease-related therapeutics, a beauty care-related component (e.g., an anti-wrinkle agent, a skin-aging inhibitor and a skin whitening agent), but not limited to.

The above-mentioned biodrug may be insulin, IGF-1 (insulin-like growth factor 1), growth hormone, erythropoietin, G-CSFs (granulocyte-colony stimulating factors), GM-CSFs (granulocyte/macrophage-colony stimulating factors), interferon-a, interferon-β, interferon-γ, interleukin-1α and 1β, interleukin-3, interleukin-4, interleukin-6, interleukin-2, EGFs (epidermal growth factors), calcitonin, ACTH (adrenocorticotropic hormone), TNF (tumor necrosis factor), atobisban, buserelin, cetrorelix, deslorelin, desmopressin, dynorphin A (1-13), elcatonin, eleidosin, eptifibatide, GHRH-II (growth hormone releasing hormone-II), gonadorelin, goserelin, histrelin, leuprorelin, lypressin, octreotide, oxytocin, pitressin, secretin, sincalide, terlipressin, thymopentin, thymosine al, triptorelin, bivalirudin, carbetocin, cyclosporin, exedine, lanreotide, LHRH (luteinizing hormone-releasing hormone), nafarelin, parathyroid hormone, pramlintide, T-20 (enfuvirtide), thymalfasin, ziconotide, RNA, DNA, cDNA, antisense oligonucleotide and siRNA, but is not limited to.

The target binding region I and/or target binding region II may include an amino acid sequence capable of binding to various targets. The material to be targeted by the bipodal-peptide binder includes a biological target such as a biochemical material, a peptide, a polypeptide, a nucleic acid, a carbohydrate, a lipid, a cell and a tissue, a compound, a metal material or a non-metal material, and preferably, a biological target.

Preferably, the biological target to be bound with the target binding region includes a biochemical material, a peptide, a polypeptide, a glycoprotein, a nucleic acid, a carbohydrate, a lipid or a glycolipid.

For instance, a biochemical material to be bound with the target binding region includes various in vivo metabolites (e.g., ATP, NADH, NADPH, carbohydrate metabolite, lipid metabolite and amino acid metabolite).

An illustrative example of peptide or polypeptide to be bound with the target binding region includes, but is not limited to, an enzyme, a ligand, a receptor, a biomarker, a hormone, a transcription factor, a growth factor, an immunoglobulin, a signal transduction protein, a binding protein, an ionic channel, an antigen, an attachment protein, a structure protein, a regulatory protein, a toxic protein, a cytokine and a coagulation factor. In more detail, a target of a bipodal-peptide binder includes fibronectin extra domain B (ED-B), VEGF (vascular endothelial growth factor), VEGFR (vascular endothelial growth factor receptor), VCAM1 (vascular cell adhesion molecule-1), nAchR (Nicotinic acetylcholine receptor), HAS (Human serum albumin), MyD88, EGFR (Epidermal Growth Factor Receptor), HER2/neu, CD20, CD33, CD52, EpCAM (Epithelial Cell Adhesion Molecule), TNF-α (Tumor Necrosis Factor-α), IgE (Immunoglobulin E), CD11A (α-chain of lymphocyte function-associated antigen 1), CD3, CD25, Glycoprotein IIb/IIIa, integrin, AFP (Alphα-fetoprotein), β2M (Beta2-microglobulin), BTA (Bladder Tumor Antigens), NMP22, cancer antigen 125, cancer antigen 15-3, calcitonin, carcinoembryonic Antigen, chromogranin A, estrogen receptor, progesterone receptor, human chorionic gonadotropin, neuron-specific enolase, PSA (Prostate-Specific Antigen), PAP (Prostatic Acid Phosphatase) and thyroglobulin.

An exemplified example of nucleic acid molecule to be bound with the target binding region includes, but is not limited to, gDNA, mRNA, cDNA, rRNA (ribosomal RNA), rDNA(ribosomal DNA) and tRNA. An illustrative example of carbohydrate to be bound with the target binding region includes cellular carbohydrates such as monosaccharides, disaccharides, trisaccharides and polysaccharides, but is not limited to. An exemplified example of lipid to be bound with the target binding region includes fatty acid, triacylglycerol, sphingolipid, ganglioside and cholesterol, but is not limited to.

The bipodal-peptide binder of the present invention may be bound to not only a biomolecule (e.g., protein) exposed on a cell surface but also an intracellular biomolecule (e.g., protein) to regulate activities of biomolecules.

Where the bipodal-peptide binder targets to intracellular proteins, it is preferable that the bipodal-peptide binder further comprises a cell penetrating peptide (CPP).

The above-described CPP includes various CPPs known to those ordinarily skilled in the art, for example, HIV-1 Tat protein, oligoarginine, ANTP peptide, HSV VP22 transcription modulating protein, MTS peptide derived from vFGF, Penetratin,

Transportan, Pep-1 peptide, Pep-7 peptide, Buforin II, MAP (model amphiphatic peptide), k-FGF, Ku 70, pVEC, SynB1 and HN-1, but not limited to. The CPP may be linked to the bipodal-peptide binder according to various methods known to those skilled in the art, for example covalently binding CPP to a lysine residue of loop region in the structure stabilizing region of the bipodal-peptide binder.

There are numerous target proteins which play a critical function in in vivo physiological activity, and the bipodal-peptide binder linked to CPP is penetrated into a cell and bound to these target proteins, contributing to regulation (e.g., suppression) of their activities.

As described above, the bipodal-peptide binder of the present invention has a “N-target binding region I-one strand of structure stabilizing region-the other strand of structure stabilizing region-target binding region II-C” construct.

According to a preferable embodiment, the bipodal-peptide binder of the present invention includes a structure influence inhibiting region which blocks a structural interaction between target binding region and structure stabilizing region and is located at an interspace between target binding region I and one strand of structure stabilizing region and/or between and the other strand of structure stabilizing region and target binding region II. Rotation region of peptide molecule includes an amino acid which φ and ψ rotation are relatively free in peptide molecule. Preferably, an amino acid which φ and ψ rotation are relatively free is glycine, alanine and serine. The number of amino acid in the structure influence inhibiting region of the present invention may be used in a range of 1-10, preferably 1-8 and more preferably 1-3.

A library of the bipodal-peptide binder of the present invention having the above-described construct may be obtained according to various methods known in the art. The bipodal-peptide binder in the library has random sequence. The term “random sequence” used herein means that no sequence preference or no determined (or fixed) amino acid sequence is placed at any position of target binding region I and/or target binding region II. For example, the library of the bipodal-peptide binder may be constructed according to split-synthesis method (Lam et al. (1991) Nature 354:82; WO 92/00091) which is carried out on solid supporter (e.g., polystyrene or polyacrylamide resin).

According to a preferable embodiment, the library of the bipodal-peptide binder is constructed by a cell surface display method (e.g., phage display, bacteria display or yeast display). Preferably, the library of the bipodal-peptide binder is prepared by a display method based on plasmids, bacteriophages, phagemids, yeasts, bacteria, mRNAs or ribosomes.

Phage display is a technique displaying various polypeptides as proteins fused with coat protein on phage surface (Scott, J. K. and Smith, G. P. (1990) Science 249: 386; Sambrook, J. et al., Molecular Cloning. A Laboratory Manual, 3rd ed. Cold Spring Harbor Press (2001); Clackson and Lowman, Phage Display, Oxford University Press (2004)). Gene of interest is fused with gene III or gene VIII of filamentous phage (e.g., M13), thereby displaying random peptides.

Phagemid may be utilized in phage display. Phagemid is a plasmid vector which has a replication origin of bacteria (e.g., ColE1) and one copy of intergenic region of bacteriophage. DNA fragment cloned into the phagemid is proliferated as same as a plasmid.

Using a phage display method for constructing a library of a bipodal-peptide binder, a preferable embodiment of the present invention includes the steps of: (i) preparing a library of an expression vector including a fusion gene in which a gene encoding a phage coat protein (e.g., gene III or gene VIII coat protein of filamentous phage such as M13) is fused with a gene encoding a bipodal-peptide binder, and a transcriptional regulatory sequence (e.g., lac promoter) operatively linked to the fusion gene; (ii) introducing the library into a suitable host cell; (iii) displaying a fusion protein on the phage surface by culturing the host cell and forming a recombinant phage or a phagemid virus particle; (iv) binding the particle to a target molecule by contacting the virus particle with a biological target molecule; and (v) removing the particle unbound to the target molecule.

The method to construct and screen a peptide library using a phage display method is disclosed in U.S. Pat. Nos. 5,723,286, 5,432,018, 5,580,717, 5,427,908, 5,498,530, 5,770,434, 5,734,018, 5,698,426, 5,763,192 and 5,723,323.

The method to prepare an expression vector including a bipodal-peptide binder may be carried out according to the method known in the art. For example, expression vector may be prepared by inserting a bipodal-peptide binder into a public phagemid or phage vector (e.g., pIGT2, fUSES, fAFF1, fd-CAT1, m663, fdtetDOG, pHEN1, pComb3, pComb8, pCANTAB 5E (Pharmacia), LamdaSurfZap, pIF4, PM48, PM52, PM54, fdH and p8V5).

Most phage display methods are carried out using filamentous phage. Additionally, a library of bipodal-peptide binder may be constructed using lambda phage display (WO 95/34683; U.S. Pat. No. 5,627,024), T4 phage display (Ren et al. (1998) Gene 215:439; Zhu (1997) CAN 33:534) and T7 phage display (U.S. Pat. No. 5,766,905).

The method to introduce a vector library into a suitable host cell may be performed according to various transformation methods, and most preferably, electroporation (See, U.S. Pat. Nos. 5,186,800, 5,422,272 and 5,750,373). The host cell suitable in the present invention includes gram-negative bacteria such as E. coli which includes JM101, E. coli K12 strain 294, E. coli strain W3110 and E. coli XL-1Blue (Stratagene), but is not limited to. It is preferable that host cells are prepared as a competent cell before transformation (Sambrook, J. et al., Molecular Cloning. A Laboratory Manual, 3rd ed. Cold Spring Harbor Press(2001)). In general, selection of transformed cells may be carried out by culturing cells in a medium containing antibiotics (e.g., tetracycline and ampicillin). Selected transformants are further cultured in the presence of helper phage to produce recombinant phages or phagemid virus particles. Suitable helper phage as described above includes, but is not limited to, Ex helper phage, Mβ-K07, Mβ-VCS and R408.

Selection of virus particle binding to a biological target molecule may be carried out using a conventional biopanning process (Sambrook, J. et al., Molecular Cloning. A Laboratory Manual, 3rd ed. Cold Spring Harbor Press(2001); Clackson and Lowman, Phage Display, Oxford University Press(2004)).

Practical example of the bipodal-peptide binder of the present invention is described in SEQ ID NOs: 20-38 and SEQ ID NOs: 40-41.

In still another aspect of this invention, there is provided a nucleic acid molecule encoding the intracellular targeting bipodal-peptide binder of the present invention.

In still another aspect of this invention, there is provided a vector for expressing the intracellular targeting bipodal-peptide binder including the nucleic acid molecule encoding the intracellular targeting bipodal-peptide binder.

In further still another aspect of this invention, there is provided a transformant containing the vector for expressing the intracellular targeting bipodal-peptide binder.

The term “nucleic acid molecule” as used herein refers to a comprehensive DNA (gDNA and cDNA) and RNA molecule, and a nucleotide as a basic unit in the nucleic acid includes not only natural nucleotides but also analogues which a sugar or base are modified (Scheit, Nucleotide Analogs, John Wiley, New York (1980); Uhlman and Peyman, Chemical Reviews, 90:543-584 (1990)).

According to a preferable embodiment, the vector of the present invention includes not only the nucleic acid molecule encoding a bipodal-peptide binder but also a strong promoter (e.g., tac promoter, lac promoter, lacUV5 promoter, lpp promoter, p_{L}^{λ}promoter, p_{R}^{λ}promoter, racy promoter, amp promoter, recA promoter, SP6 promoter, trp promoter and T7 promoter, etc.) for transcription, a ribosome-binding site for translation, and transcription/translation termination sequence.

According to a preferable embodiment, the vector of the present invention further includes a signal sequence (e.g., pelB) at 5′-end of nucleic acid molecule encoding a bipodal-peptide binder. According to a preferable embodiment, the vector of the present invention further includes a tagging sequence (e.g., myc tag) to examine whether bipodal-peptide binder is suitably expressed on phage surface.

According to a preferable embodiment, the vector of the present invention includes a phage coat protein, preferably a gene encoding a gene III or gene VIII coat protein of filamentous phage such as M13. According to a preferable embodiment, the vector of the present invention includes a replication origin of bacteria (e.g., ColE1) and/or bacteriophage. In addition, the vector of the present invention includes an antibiotics-resistance gene known to those ordinarily skilled in the art as a selection marker, for example resistant genes against ampicillin, gentamycin, carbenicillin, chloramphenicol, streptomycin, kanamycin, geneticin, neomycin and tetracycline.

The transformant of the present invention preferably includes gram-negative bacteria such as E. coli which includes JM101, E. coli K12 strain 294, E. coli strain W3110 and E. coli XL-1Blue (Stratagene), but is not limited to. The procedure to deliver the present vector into a cell may be carried out according to various methods known to those ordinarily skilled in the art. For example, the transformation using a prokaryotic cell as a host may be performed according to a CaCl₂ method (Cohen, S. N. et al., Proc. Natl. Acac. Sci. USA, 9: 2110-2114 (1973)), a Hanahan method (Cohen, S. N. et al., Proc. Natl. Acac. Sci. USA, 9:2110-2114 (1973); and Hanahan, D., J. Mol. Biol., 166: 557-580 (1983)) and an electroporation method (U.S. Pat. Nos. 5,186,800, 5,422,272 and 5,750,373).

The bipodal-peptide binder of the present invention exhibits the K_{D} value (dissociation constant) of a very low level (for example, nM level) and, therefore, exhibits very high affinity toward a biological target molecule. As described in Examples below, the bipodal-peptide binder has about 10²-10⁵-fold (preferably, about 10³-10⁴-fold) affinity higher than a monopodal peptide binder. The bipodal-peptide binder of the present invention has applications not only in pharmaceuticals and detection of in vivo material but also in in vivo imaging, in vitro cell imaging, and drug delivery targeting, and can be very usefully employed as an escort molecule.

### Advantegeous Effects of the Invention

The features and advantages of the present invention will be summarized as follows:

(i) The present invention provides a BPB-based cargo delivery system.

(ii) The distal two target binding regions which are linked to each both termini of a structure stabilizing region in the bipodal-peptide binder of the present invention bind in a cooperative or synergetic manner to the target.

(iii) In this connection, the bipodal-peptide binder of the present invention exhibits the K_{D} value (dissociation constant) of a very low level (for example, nM level) and, therefore, exhibits very high affinity toward a biological target molecule.

(iv) The BPB-based cargo delivery system permits to deliver various materials into cells or to cell surface with the help of target binding ability and specificity of the BPB.

### BRIEF DESCRIPTION OF THE DRAWINGS

FIG. 1a schematically represents a bipodal-peptide binder containing a β-hairpin as a structure stabilizing region.

FIG. 1b schematically represents a bipodal-peptide binder containing a β-sheet linked by a linker as a structure stabilizing region.

FIG. 1c schematically represents a bipodal-peptide binder containing a leucine zipper motif linked by a linker as a structure stabilizing region.

FIG. 1d schematically represents a bipodal-peptide binder containing a leucine-rich motif linked by a linker as a structure stabilizing region.

FIG. 2 shows a strategy for cloning a bipodal-peptide binder library. In a map of pIGT2 phagemid vector, a pelB signal sequence and myc tag are tagging sequences to determine whether a gene of interest is suitably expressed on phage surface. lac promoter was used as a promoter.

FIG. 3 is a biopanning result of ED-B, streptavidin and BSA to input phage in fibronectin ED-B biopanning process.

FIG. 4 represents ELISA to ED-B and BSA of 60 recombinant phages recovered from third biopanning of a bipodal-peptide binder library in fibronectin ED-B biopanning process.

FIG. 5a is a result to monitor an affinity of the bipodal-peptide binder of the present invention to be specifically bound to fibronectin ED-B.

FIG. 5b shows a result to monitor an affinity of the bipodal-peptide binder of the present invention to be specifically bound to VEGF.

FIG. 5c represents a result to monitor an affinity of the bipodal-peptide binder of the present invention to be specifically bound to VCAM1.

FIG. 5d shows a result to monitor an affinity of the bipodal-peptide binder of the present invention to be specifically bound to nAchR (Nicotinic acetylcholine receptor).

FIG. 5e is a result to measure an affinity of the bipodal-peptide binder of the present invention to be specifically bound to HAS (Human Serum Albumin).

FIG. 6a is a graph to measure absorbance through ELISA against several proteins using a recombinant phage containing the bipodal-peptide binder of the present invention to examine specificity to fibronectin ED-B. X axis is in a order of streptavidin, ED-B, acetylcholine α1 (a1), BSA, VCAM, TNF-α, thrombin, myoglobin, lysozyme and visfatin from the left bar.

FIG. 6b shows a graph to measure absorbance through ELISA against several proteins using a recombinant phage containing the bipodal-peptide binder of the present invention to examine specificity to VEGF.

FIG. 6c represents a graph to measure absorbance through ELISA against several proteins using a recombinant phage containing the bipodal-peptide binder of the present invention to examine specificity to VCAM1.

FIG. 6d is a graph to measure absorbance through ELISA against several proteins using a recombinant phage containing the bipodal-peptide binder of the present invention to examine specificity to nAchR.

FIG. 6e represents a graph to measure absorbance through ELISA against several proteins using a recombinant phage containing the bipodal-peptide binder of the present invention to examine specificity to HSA.

FIG. 6f shows a graph to measure absorbance through ELISA against several proteins using a recombinant phage containing the bipodal-peptide binder of the present invention to examine specificity to MyD88.

FIG. 7 is a result to monitor an affinity for verifying a cooperative binding activity of the bipodal-peptide binder of the present invention.

FIG. 8 shows a result to monitor an affinity of the bipodal-peptide binder of the present invention by replacing tryptophan zipper motif with several β-hairpin motifs as a structure stabilizing region in the bipodal-peptide binder.

FIG. 9 represents a result to monitor an affinity of the bipodal-peptide binder of the present invention by replacing tryptophan zipper motif with a leucine zipper motif as a structure stabilizing region in the bipodal-peptide binder.

FIG. 10 represents a cancer targeting of the bipodal-peptide binder of the present invention specific to fibronectin ED-B as a cancer biomarker. It was shown that the bipodal-peptide binder is accumulated in a tumor portion of mouse with the passage of time. In addition, it was observed that the bipodal-peptide binder is significantly accumulated in each internal organ (e.g., liver, heart, lung, kidney, spleen, etc.) through fluorescence measurement.

FIG. 11a represents purification of the GST-BPB fusion protein.

FIG. 11b represents affinity of the GST-BPB to fibronectin EDB.

FIG. 12a represents purification of the TNFα-BPB.

FIG. 12b represents affinity of the TNFα-BPB to fibronectin EDB.

FIG. 12c represents cytotoxicity of the TNFα-BPB to fibronectin EDB.

FIG. 13a represents analysis results of encapsulation efficiency of 9R/siRNA into BPB_{css}-LS. The green box denotes a liposomal fraction containing the 9R/siRNA complex and the red box denotes free unencapsulated siRNA fraction.

FIG. 13b represents uptake of BPB_{css}-LS in EDB over-expressing cell lines. Negative corresponds to untreated cells, LS to cells treated only with lipolsome and CSS-LS to cells treated with BPB_{css}-LS.

FIG. 13c represents uptake of BPB_{css}-LS in EDB over-expressing cell lines. Negative corresponds to untreated cells, LS to cells treated only with lipolsome and CSS-LS to cells treated with BPB_{css}-LS.

FIG. 13d represents VEGF-C siRNA knockdown efficiency in MCF-7 cells.

FIG. 14a represents MALDI analysis results for conjugation between DSPE-PEG₂₀₀₀-Mal and BPB(SSS) peptide.

FIG. 14b represents ELS analysis results to investigate change in hydrodynamic size of SPION-BPB.

FIG. 14c represents TEM images of (a) DSPE-PEG2000 coated SPION and (b) BPB conjugated DSPE-PEG2000 coated SPION.

FIG. 14d represents results of T2-weighted MR phantom study of DSPE-PEG2000 coated SPION (DSPE-SPION) and BPB conjugated DSPE-PEG2000 coated SPION (SSS-SPION).

FIG. 14e represents (a) MRI images and (b) T2 signal of cells treated with either DSPE-PEG2000 coated SPION or BPB conjugated DSPE-PEG2000 coated SPION.

FIG. 14f represents confocal microscopic images of cells treated with either DSPE-PEG2000 coated SPION or BPB conjugated DSPE-PEG2000 coated SPION.

FIG. 14g represents MTT assay results of DSPE-PEG2000 coated SPION and BPB conjugated DSPE-PEG2000 coated SPION.

FIG. 14h represents MR images of either DSPE-PEG2000 coated SPION or BPB conjugated DSPE-PEG2000 coated SPION in brain tumor animal models.

FIG. 15a represents TEM image of gold nanoparticle-BPB.

FIG. 15b represents the amount of Au in U87MG cells treated with BPB conjugated gold nanoparticles.

FIG. 15c represents silver enhancement microscopic images of U87MG cells treated with either (a) GNP or (b) BPB-GN.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Experiment Material and Method

### Example 1

### Library Construction

### Bipodal-peptide Binder Gene Preparation and Insertion to Phagemid Vector

We synthesized two degenerate BPB-encoding oligonucleotides, BPB-F1 and BPB-B1, with the sequences 5′-TTCTATGCGGCCCAGCTGGCC (NNK)₆GGATCTTGGACATGGGAAAACGGAAAA-3′ and 5′-AACAGTTTCTGCGGCCGCTCCTCC TCC(MNN)₆TCCCTTCCATGTCCATTTTCCGTT-3, respectively, where N is A, T, G or C; K is G or T; and M is C or A (Genotech). To synthesize double strand, Beta-F1 (4 μM), Beta-B1 (4 μM), 2 μl dNTP mixture (2.5 mM), 1 μl ExTaq DNA polymerase (Takara, Seoul, Korea) and 10×PCR buffer were mixed and then distilled water was added to a final volume of 50 μl, preparing the mixture solution in total number of 25. After the double strand in the mixture was prepared by performing PCR (predenaturing step, 5 min at 94° C.; 60 cycles-30 sec at 94° C.; 30 sec at 72° C.; and 7 min at 72° C.), the purification was carried out using PCR purification kit (GeneAll, Seoul, Korea), obtaining a bipodal-peptide binder (BPB) gene. To link the gene to be inserted into bipodal-peptide binder with pIGT2 phagemid vector (Ig therapy, Chuncheon, Korea), insert gene and pIGT2 phagemid vector were restricted with restriction enzyme. About 11 μg insert DNA were restricted with Sfil (New England Biolabs(NEB, Ipswich) and Notl (NEB, Ipswich) for 4 hrs, respectively, followed by purification using PCR purification kit. In addition, About 40 μg pIGT2 phargemid vector were restricted with Sfil and Notl for 4 hrs, respectively, and then CIAP (Calf Intestinal Alkaline Phosphatase; NEB, Ipswich) was treated for 1 hr, followed by purification using PCR purification kit. Both insert DNA and pGIT2 phargemid vector were quantitated using UV-visible light spectrophotometer (Ultrospec 2100pro, Amersham Bioscience), and 2.9 μg insert DNA were ligated with 12 μg pIGT2 phargemid vector at 18° C. for 15 hrs using T4 DNA ligase (Bioneer, Daejeon, Korea). After ethanol precipitation, DNA were dissolved in 100 μl TE buffer.

### Competent Cell Preparation

E. coli XL1-BLUE (American Type Culture Collection, Manassas, USA) cells were linearly spread in LB agar-plate. The colony grown on solid agar media was inoculated into 5 ml LB media, and then incubated at 37° C. overnight with shaking at 200 rpm. The cells (10 ml) were inoculated into 2 liter of LB media, and cultured in the same manner until reaching at 0.3-0.4 of absorbance at 600 nm. The cultured flask was placed on ice for 30 min, and centrifuged at 4,000×g for 20 min at 4° C. The supernatant was completely removed, and the precipitated cells were suspended in 1 liter cold-sterile distilled water. After performing repeatedly as described above, the cells were resuspended in 1 liter cold-sterile distilled water. Also, after centrifugation and washing with 40 ml glycerol solution (10%), the cells were finally dissolved in 4 ml glycerol solution (10%) and aliquoted to 200 μl. Aliquots (200 μl) were freezed with liquid nitrogen, and stored at −80° C. until use.

### Electroporation

Electroporation was carried out using 25 aliquots of 100 μl mixture in which 2.9 pg insert DNA are linked to 12 μg phagemid vector and a bipodal-peptide binder. After competent cells (200 μl) were dissolved on ice and mixed with 4 μl aliquot, the mixture was put into 0.2 cm cuvette and placed on ice for 1 min. Using an electroporator (BioRad, Hercules, Calif.) set the resistance at 200 Ω, the capacitance at 25 μF and the voltage to 2.5 kV, electric pulse (time constant, 4.5-5 msec) is applied to the cuvette. Immediately, the mixture was added to 1 ml LB liquid media containing 20 mM glucose to be pre-warmed at 37° C., and cells in total volume of 25 ml were obtained and then transferred into 100 ml test tube. After culturing at 200 rpm for 1 h at 37° C., 10 μl diluents were spread on ampicillin-agar media plate to count the number of library. The remaining cells were cultured overnight at 30° C. in 1 liter LB containing 20 mM glucose and 50 μg/ml ampicillin. After the supernatant was completely removed by centrifugation at 4,000×g for 20 min at 4° C. and the precipitated cells were resuspended in 40 ml LB media, the cells were finally dissolved in glycerol solution of not less than 20%, and stored at −80° C. until use.

Recombinant Phage Production from Library and PEG Precipitation

Recombinant phages were prepared from a bipodal-peptide binder library stored at −80° C. After 50 μg/ml ampicillin and 20 mM glucose were added to 100 ml LB liquid media in 500 ml flask, 1 ml library stored at −80° C. were inoculated into the media and then cultured at 150 rpm for 1 hr at 37° C. Afterwards, Ex helper phages (1×10¹¹ pfu/ml; Ig therapy, Chuncheon, Korea) were added to the media and cultured for 1 hr in the equal conditions. After removing the supernatant through centrifugation at 1,000×g for 10 min, the cells were incubated overnight in 100 ml LB liquid media supplemented with 50 μg/ml ampicillin and 25 μg/ml kanamycin to produce recombinant phages. After centrifuging the culture solution at 3,000×g for 10 min, 100 ml of the supernatant were mixed with 25 ml PEG/NaCl solution and kept to stand on ice for 1 hr. The supernatant was removed by centrifuging the culture solution at 10,000×g for 20 min at 4° C., and the pellet was resuspended in 2 ml PBS (pH 7.4).

### Example 2

### Protein Preparation

Fibronectin ED-B, VEGF (vascular endothelial growth factor), VCAM1 (vascular cell adhesion molecule-1), nAchR (Nicotinic acetylcholine receptor), HAS (Human serum albumin) and MyD88 to be used in the Examples were prepared as follows.

Fibronectin ED-B Gene Construction and Insertion into Expression Vector

Partial human fibronectin ED-B (ID=KU017225) gene were provided from Korea Research Institute of Bioscience & Biotechnology (KRIBB). We synthesized two primers, EDB_F1 (5′-TTCATAACATATGCCAGAGGTGCCCCAA-3′) and EDB_B1 (5′-ATTGGATCCTTACGTTTGTTGTGTCAGTGTAGTAGGGGCACTCTCGCCGCCATTAATGAGAGTGATAACGCTGATATCATAGTCAATGCCCGGCTCCAGCCCTGTG-3′). Twenty pmol EDB_F1, 20 μmol EDB_B1, 4 μl dNTP mixture (2.5 mM), 1 μl ExTaq DNA polymerase (10 U) and 5 μl 10×PCR buffer were mixed and then distilled water was added to a final volume of 50 μl, preparing the mixture solution. After the EDB insert was prepared by performing PCR (pre-denaturing step, 5 min at 94° C.; 30 cycles-30 sec at 94° C.; 30 sec at 55° C.; and 1 min at 72° C.), and purified using PCR purification kit. To clone the insert into pET28b vector, EDB insert and pET28b vector were restricted with restriction enzyme. About 2 μg EDB insert were restricted with BamHI (NEB, Ipswich) and NdeI (NEB, Ipswich) for 4 hrs, followed by purification using PCR purification kit. In addition, About 2 μg pIGT2 phargemid vector were restricted with BamHI and NdeI for 3 hrs, respectively, and then CIAP was treated for 1 hr, followed by purification using PCR purification kit. The vector and insert were mixed at a molar ratio of 1:3 and ligated at 18° C. for 10 hrs using T4 DNA ligase (Bioneer, Daejeon, Korea). After transformation to XL-1 competent cells, the transformed cells were spread in agar media containing kanamycin. The colony grown on a solid agar plate was inoculated into 5 ml LB media, and then incubated at 37° C. overnight with shaking at 200 rpm. Plasmids were purified by plasmid preparation kit (GeneAll, Seoul, Korea), and then sequenced to determine whether the cloning is successive.

VEGF121 Gene Construction and Insertion into Expression Vector

Partial human VEGF (ID=G157) gene were provided from Bank for Cytokine Research (BCR; Jeonju, Korea). We synthesized two primers, VEGF_F1 (5′-ATAGAATTCGCACCCATGGCAGAA-3′) and VEGF_B1 (5′- ATTAAGCTTTCACCGCCTCGGCTTGTCACAATTTTCTTGTCTTGC-3′). Twenty pmol VEGF_F1, 20 μmol VEGF_B1, 4 μl dNTP mixture (2.5 mM), 1 μl ExTaq DNA polymerase (10 U) and 5 μl 10×PCR buffer were mixed and then distilled water was added to a final volume of 50 μl, preparing the mixture solution. After the VEGF insert was prepared by performing PCR (pre-denaturing step, 5 min at 94° C.; 30 cycles-30 sec at 94° C.; 30 sec at 55° C.; and 1 min at 72° C.), and purified using PCR purification kit. To clone the insert into pET32a vector (Novagen), VEGF insert and pET32a vector were restricted with restriction enzyme. About 2 μg VEGF insert were restricted with EcoRI (NEB, Ipswich) and HindIII (NEB, Ipswich) for 4 hrs, followed by purification using PCR purification kit. The vector and insert were mixed at a molar ratio of 1:3 and ligated at 18° C. for 10 hrs using T4 DNA ligase (Bioneer, Daejeon, Korea). After transformation to XL-1 competent cells, the transformed cells were spread in agar media containing ampicillin. The colony grown on a solid agar plate was inoculated into 5 ml LB media, and then incubated at 37° C. overnight with shaking at 200 rpm.

Plasmids were purified by plasmid preparation kit (GeneAll, Seoul, Korea), and then sequenced to determine whether the cloning is successive.

VCAM1 Gene Construction and Insertion into Expression Vector

Human VCAM gene was provided from Korea Research Institute of Bioscience & Biotechnology (KRIBB). To clone the insert into pET32a vector, VCAM1 insert and pET32a vector were restricted with restriction enzyme. The vector and insert were mixed at a molar ratio of 1:3 and ligated at 18° C. for 10 hrs using T4 DNA ligase (Bioneer, Daejeon, Korea). After transformation to XL-1 competent cells, the transformed cells were spread in agar media containing ampicillin. The colony grown on a solid agar plate was inoculated into 5 ml LB media, and then incubated at 37° C. overnight with shaking at 200 rpm. Plasmids were purified by plasmid preparation kit (GeneAll, Seoul, Korea), and then sequenced to determine whether the cloning is successive.

### Expression and Purification Fibronectin ED-B

After transformation of pET28b vector carrying fibronectin ED-B into BL21 cells, the transformed cells were spread in agar media containing kanamycin. The colony grown on a solid agar plate was inoculated into 5 ml LB media containing kanamycin (25 μg/ml), and then incubated at 37° C. overnight with shaking at 200 rpm, followed by further incubation for 3 hrs in 50 ml of fresh LB media containing kanamycin (25 μg/ml). The cultured E coil were inoculated into 2 liter of LB containing kanamycin (25 μg/ml) and then cultured to OD=0.6-0.8. Afterwards, 1 mM isopropyl-β-D-thiogalactopyranoside (IPTG) were added to the media and cultured at 37° C. for 8 hrs with shaking at 200 rpm. After removing the supernatant through centrifugation at 4,000×g for 20 min, the precipitated cells were suspended in lysis buffer [50 mM sodium phosphate (pH 8.0), 300 mM NaCl and 5 mM imidazole]. After storing at −80° C. overnight, E. coli were lysed using a sonicator and then centrifuged at 15,000×g for 1 hr, followed by binding the supernatant to Ni-NTA affinity resin (Elpisbio, Daejeon, Korea). After washing the resin with lysis buffer, N-terminal His-tag ED-B proteins were eluted with elution buffer [50 mM sodium phosphate (pH 8.0), 300 mM NaCl and 300 mM imidazole]. ED-B protein with high purity was obtained from the eluent by gel filtration using Superdex75 column (GE Healthcare, United Kingdom) and PBS (pH 7.4). For biopanning, biotin is conjugated to the ED-B protein. Six mg of sulfo-NHS-SS-biotin (PIERCE, Illinois, USA) and 1.5 mg ED-B protein were incubated in 0.1 M sodium borate (pH 9.0) at room temperature for 2 hrs. To eliminate residual sulfo-NHS-SS-biotin, biotinylated-EDB protein was purified by gel filtration using Superdex75 column and PBS (pH 7.4).

Expression and Purification of VEGF121 and VCAM1-Trx

After transformation of pET32a vector carrying VEGF121 and VCAM1 into AD494 cells, the transformed cells were spread in agar media containing ampicillin, respectively. The colony grown on a solid agar plate was inoculated into 5 ml LB media containing ampicillin (25 μg/ml), and then incubated at 37° C. overnight with shaking at 200 rpm, followed by further incubation for 3 hrs in 50 ml of fresh LB media containing ampicillin (25 μg/ml). The cultured E co/iwere inoculated into 2 liter of LB containing kanamycin (25 μg/ml) and then cultured to OD=0.6-0.8. Afterwards, 1 mM isopropyl-β-D-thiogalactopyranoside (IPTG) were added to the media and cultured at 37° C. for 8 hrs with shaking at 200 rpm. After removing the supernatant through centrifugation at 4,000×g for 20 min, the precipitated cells were suspended in lysis buffer [50 mM sodium phosphate (pH 8.0), 300 mM NaCl and 5 mM imidazole]. After storing at −80° C. overnight, E. coli were lysed using a sonicator and then centrifuged at 15,000×g for 1 hr, followed by binding the supernatant to Ni-NTA affinity resin (Elpisbio, Daejeon, Korea). After washing the resin with lysis buffer, Trx-VEGF121 and Trx-VCAM1 proteins were eluted with elution buffer [50 mM sodium phosphate (pH 8.0), 300 mM NaCl and 300 mM imidazole]. VEGF-Trx and VCAM1-Trx protein with high purity were obtained from the eluent by gel filtration using Superdex75 column (GE Healthcare, United Kingdom) and PBS (pH 7.4). For obtaining pure VEGF121 protein, VEGF-Trx was cut with thrombin.

Meanwhile, HAS was purchased from Genetex Inc. (Irvine). Biotin-SGEWVIKEARGWKHWVFYSCCPTTPYLDITYH (32 mer), a peptide fragment of nAchR (Nicotinic acetylcholine receptor), was synthesized from Anigen Inc. (Korea, Kwangju). Human MyD88 was purchased from Santa Cruz Biotechnology (sc-4540 WB; California).

### Example 3

### Biopanning

Biopanning of Biotinylated-Fibronectin ED-B protein and Biotinylated-nAchR Peptide

Two ml of straptavidin (10 μg/ml) were added to 40 wells (50 μl per well) in a 96-well ELISA plate and then kept to stand at 4° C. overnight. Next day, only 20 wells were washed with 0.1% PBST (tween-20) three times, and each biotinylated ED-B and biotinylated nAchR (10 μg/ml) was added and incubated at room temperature for 1 hr. Afterwards, all 40 wells were washed with 0.1% PBST (tween-20) three times and blocked at room temperature for 2 hrs using 2% BSA diluted with PBS. Then, the solution was removed and the plate was washed with 0.1% PBST three times. To eliminate streptavidin- and BSA-bound phages, the mixture of 800 μl solution containing bipodal-peptide binder recombinant phages and 200 μl BSA (10%) was added to 20 wells coated with streptavidin and BSA, and incubated at 27° C. for 1 hr. The supernatant collected was transferred to the well in which ED-B and nAchR was bound, and kept to stand at 27° C. for 45 min. The solution in 20 wells was completely removed and washed with 0.5% PBST 15 times in round 1. Bound phages were subsequently eluted for 20 min by adding 1 ml of 0.2 M glycine/HCl (pH 2.2) to each well (50 μl per well). The phages were collected in 1 ml tube and neutralized by adding 150 μl of 2 M Tris-base (pH 9.0). To measure the number of input and elute phage per biopanning, the phages were mixed with XL-1 BLUE cells (OD=0.7) and spread in agar plate containing ampicillin. To repeat panning, the phages were mixed with 10 ml E. coli XL1-BLUE cells and incubated at 37° C. for 1 hr with shaking at 200 rpm. After mixing with ampicillin (50 μg/ml) and 20 mM glucose, Ex helper phages (2×10¹⁰ pfu/ml) were added to the media and cultured at 37° C. for 1 hr with shaking at 200 rpm. After removing the supernatant through centrifugation at 1,000×g for 10 min, the precipitated cells were incubated at 37° C. overnight with shaking at 200 rpm in 40 ml LB liquid media supplemented with 50 μg/ml ampicillin and 25 μg/ml kanamycin. After centrifuging the culture solution at 4,000×g for 10 min at 4° C., the supernatant were mixed with 8 ml of 5×PEG/NaCl solution [20(w/v)% PEG and 15(w/v)% NaCl] and kept to stand at 4° C. for 1 hr. The supernatant was completely removed and the phage peptide pellet was resuspended in 1 ml PBS solution, which is used in 2^{nd} biopanning. Each biopanning step was carried out according to the same method as described above except for washing with 0.1% PBST 25 times in round 2 and 35 times in round 3.

### Biopanning of VEGF and VCAM1-Trx and Human Serum Albumin (HSA), MyD88

VEGF and VCAM1-Trx and HSA and MyD88 (5 μg/ml) were added to 10 wells (50 μl per well) in a 96-well ELISA plate (Corning) and then kept to stand at 4° C. overnight. Next day, the wells were blocked at room temperature for 2 hrs with 2% BSA. Then, the solution was removed and the plate was washed with 0.1% PBST three times. The mixture of 800 μl solution containing bipodal-peptide binder recombinant phages and 200 μl BSA (10%) was added to 10 wells which VEGF and VCAM1-Trx and HSA were bound, and incubated at room temperature for 1 hr. The solution in 10 wells was completely removed and washed with 0.1% PBST 10 times in round 1. Bound phages were subsequently eluted for 20 min by adding 1 ml of 0.2 M glycine/HCl (pH 2.2) to each well (50 μl per well). The phages were collected in 1 ml tube and neutralized by adding 150 μl of 2 M Tris-base (pH 9.0). To measure the number of input and elute phage per biopanning, the phages were mixed with XL-1 BLUE cells (OD=0.7) and spread in agar plate containing ampicillin. To repeat panning, the phages were mixed with 10 ml E. coli XL1-BLUE cells and incubated at 37° C. for 1 hr with shaking at 200 rpm. After mixing with ampicillin (50 μg/ml) and 20 mM glucose, Ex helper phages (2×10¹⁰ pfu/ml) were added to the media and cultured at 37° C. for 1 hr with shaking at 200 rpm. After removing the supernatant through centrifugation at 1,000×g for 10 min, the precipitated cells were incubated at 37° C. overnight with shaking at 200 rpm in 40 ml LB liquid media supplemented with 50 μg/ml ampicillin and 25 μg/ml kanamycin. After centrifuging the culture solution at 4,000×g for 10 min at 4° C., the supernatant were mixed with 8 ml of 5×PEG/NaCl solution [20(w/v)% PEG and 15(w/v)% NaCl] and kept to stand at 4° C. for 1 hr. The supernatant was completely removed and the phage peptide pellet was resuspended in 1 ml PBS solution, which is used in 2^{nd} biopanning. Each biopanning step was carried out according to the same method as described above except for washing with 0.1% PBST 20 times in round 2 and 30 times in round 3.

### Example 4

### ELISA of Input Phage to Fibronectin ED-B

To investigate specificity, ELISA of each input phage of bipodal-peptide binder library was carried out for streptavidin, BSA and ED-B. Each straptavidin (10 μg/ml) and BSA (10 μg/ml) was added to 18 wells (50 μl per well) and 9 wells (50 μl per well) in a 96-well ELISA plate and then kept to stand at 4° C. overnight. Next day, only 9 wells of 18 wells containing streptavidin were washed with 0.1% PBST (tween-20) three times, and biotinylated ED-B (10 μg/ml) was added and incubated at room temperature for 1 hr. Afterwards, all wells were washed with 0.1% PBST (tween-20) three times and blocked at room temperature for 2 hrs using 2% BSA diluted with PBS. Then, the solution was removed and the plate was washed with 0.1% PBST three times. Each 800 μl of first, second and third phage solution containing bipodal-peptide binder recombinant phages and 200 μl BSA (10%) was mixed. Then, 100 μl of mixture was added to 3 wells coated with ED-B, streptavidin and BSA, respectively, and incubated at 27° C. for 1.5 hrs. After washing with 0.1% PBST 10 times, HRP-conjugated anti-M13 antibodies (1:1,000 dilution; GE Healthcare) were added to each well and incubated at 27° C. for 1 hr. After washing with 0.1% PBST 5 times, 100 μl tetramethylbenzidine (TMB; BD Science) as a substrate of peroxidase was seeded into each well to induce colorimetric reaction, followed by stopping the reaction adding 100 μl of 1 M HCl. The absorbance was measured at 450 nm.

Example 5

### Detection of Phage Peptide Specific to Fibronectin ED-B, VEGF, VCAM1, nAchR, HAS and MyD88 protein (Phage ELISA)

XL1-BLUE cells were transformed with phages recovered from biopanning step having the highest ratio of output phage to input phage, and spread in plate to produce 100-200 of plaques. Using a sterile tip, 60 plaques were inoculated in 2 ml LB-ampicillin (50 μg/ml) media and cultured at 37° C. for 5 hr with vigorous shaking.

The transformed cells were infected with Ex helper phages (5x10⁹ pfu/ml; OD=0.8-1.0) and cultured at 37° C. for 1 hr with shaking at 200 rpm. After removing the supernatant by centrifuging at 1,000×g for 10 min, the precipitated cells were resuspended in 1 ml LB liquid media supplemented with 50 μg/ml ampicillin and 25 pg/ml kanamycin, and cultured at 30° C. overnight with shaking at 200 rpm. The supernatant was collected by centrifuging at 10,000×g for 20 min at 4° C. and mixed with 2% skim milk, which is used in detection of phage peptides.

Fibronectin ED-B, VEGF, VCAM1, Nicotinic acetylcholine receptor (nAchR), Human serum albumin and MyD88 (each 5 μg/ml) and BSA (10 μg/ml) were added to 30 wells (50 μl per well) in a 96-well ELISA plate and then kept to stand at 4° C. overnight. Next day, all wells were washed with 0.1% PBST three times, and blocked at room temperature for 2 hrs using 2% skim milk diluted with PBS. Then, the solution was removed and the plate was washed with 0.1% PBST three times. Phage peptide solution (100 μl) amplified from each clone was divided into all wells and kept to stand at 27° C. for 1.5 hrs. After washing with 0.1% PBST 5 times, HRP-conjugated anti-M13 antibodies (1:1,000 dilution; GE Healthcare) were added to each well and incubated at 27° C. for 1 hr. After washing with 0.1% PBST 5 times, 100 μl TMB was divided into each well to induce colorimetric reaction, followed by stopping the reaction adding 100 pl of 1 M HCl. The absorbance was measured at 450 nm to select phages which had the absorbance higher than BSA. XL1 cells were infected with these phages and spread in plate to produce 100-200 of plaques. Using a sterile tip, plaques were inoculated in 4 ml LB-ampicillin (50 μg/ml) media and cultured at 37° C. overnight with vigorous shaking. Plasmids were purified by plasmid preparation kit (GeneAll, Seoul, Korea), and then sequenced. The following phagemid sequence was used for sequencing: 5′-GATTACGCCAAGCTTTGGAGC-3′.

### Example 6: Phage Peptide Specific to Fibronectin ED-B, VEGF or nAchR Binding Assay

Bipodal-peptide binder peptides specific to ED-B, VEGF or nAchR which were repetitively found in DNA sequencing were synthesized from Anigen Inc. (Korea). Affinity was measured using BIAcore X instrument (Biacore AB, Uppsala, Sweden). ED-B and nAchR were immobilized on streptavidin (SA) chip (Biacore) by injecting 2,000 RU biotinylated-EDB. VEGF was immobilized on CM5 chip (Biacore) using EDC/NHS. PBS (pH 7.4) was used as a running buffer. Kinetics at different concentrations was measured under a flow rate of 30 μl/min, and affinity was calculated using BIAevaluation software (Biacore AB, Uppsala, Sweden).

### Example 7: Cancer Targeting of Bipodal-peptide Binder Specific to Fibronectin ED-B as a Cancer Biomarker

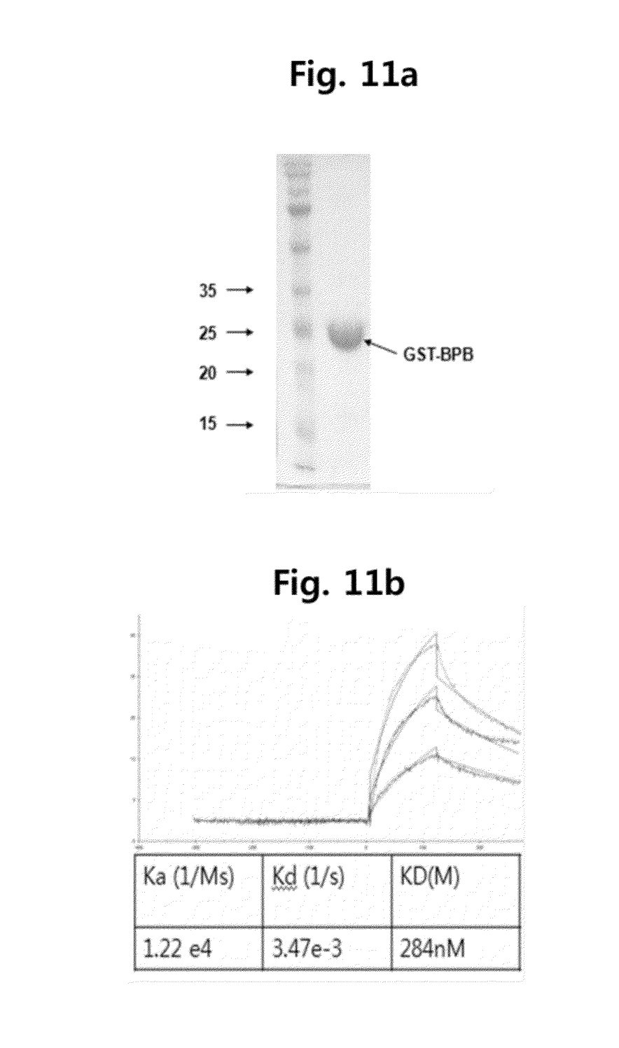

Cy5.5-NHS fluorescence dye (Amersham Pharmacia, Piscataway) was incubated in 50 mM sodium borate buffer (pH 9.7) at room temperature for 12 hrs with bipodal-peptide binder (peptide 2) which targets fibronectin ED-B widely distributed in cancer cells. After reaction, Cy5.5 and bipodal-peptide binder-Cy5.5 were separated by Sephadex G25 (Pharmacia Biotech, Uppsala, Sweden). Balb/c nude mice (Orient Bio) received subcutaneous injections of 2×10⁶ human U87MG cells (ATCC) and bred for 10 days. Subsequently, mice were intravenously injected with 0.5 nmol bipodal-peptide binder-Cy5.5 and the fluorescence was measured using IVIS (Caliper Life Sience, Hopkinton). This experiment suggests that the bipodal-peptide binder specific to ED-B as a cancer biomarker is accumulated in cancerous tissue of in vivo animal model, demonstrating its application as a practical cancer diagnostics (FIG. 11).

### Example 8

### Inhibition of Bipodal-peptide Binder Activity Specific to MyD88 Present in a Cell

Since MyD88 is a cellular protein, 9 arginines (Anigen, Korea) as a cell penetrating peptide were covalently linked to a lysine residue in loop of bipodal-peptide binder using EDC/NHS (Sigma) for penetration. As activation of MyD88 induces increase of MMP-13 amount, to investigate the amount of MMP-13 may determine whether activity of MyD88 is or not. The activity of MyD88 was activated by treating IL-lbeta (10 ng/ml; R&D systems, Minneapolis, MN) to chondrocytes. Next, 10 μM bipodal-peptide binder specific to MyD88 (peptide 1 in Table 3f) was treated to chondrocytes for 12 hrs, and then mRNA was extracted, followed by performing RT-PCR for MMP-13 and GAPDH. In addition, cellular proteins were obtained from chondrocytes and Western blotting was carried out using Anti-MMP13 antibody (Abcam, ab3208, Cambridge) and semi-dry transfer machine (Amersham Bioscience, Piscataway) to determine the amount of MMP-13.

### Experiment Results

### Example 9

### Construction of Bipodal-peptide Binder Library

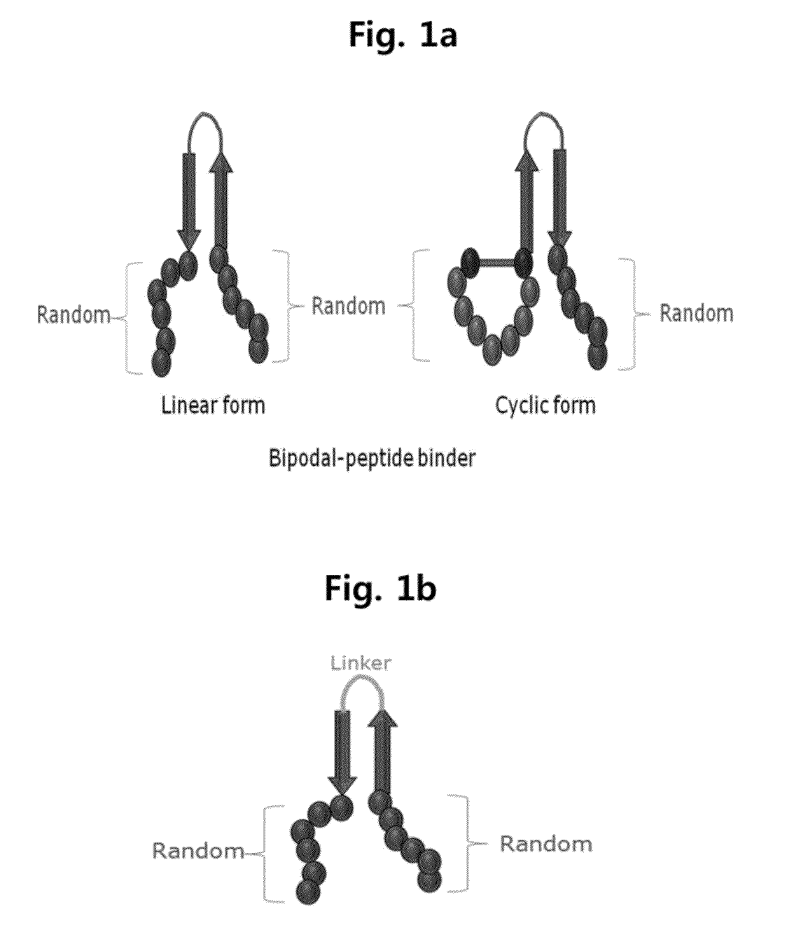

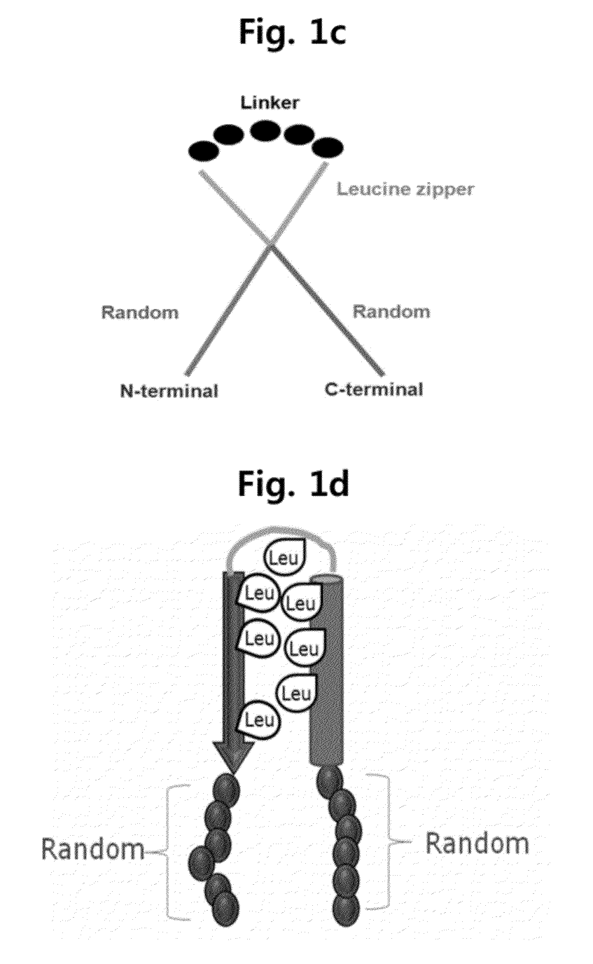

Stable β-hairpin motif was used as a structure stabilizing region of dipodal peptide binder. Given that interactions between tryptophan and tryptophan amino acids contributes to structure stability of β-hairpin motif, tryptophan (Trp) zipper motif was utilized (Andrea et al., Proc. Natl. Acad. Sci. 98:5578-5583(2001)). Each 6 amino acids in N- and C-terminal region of Trp zipper as a backbone was randomly arranged to produce variable region in both terminals (FIG. 1a). It was designated as a bipodal-peptide binder. The bipodal-peptide binder has high affinity and specificity since it binds to antibody in a cooperative manner via variable region in both termini. Additionally, the structure stabilizing region of bipodal-peptide binder may be diversely composed as demonstrated in FIGS. 1b-1e.

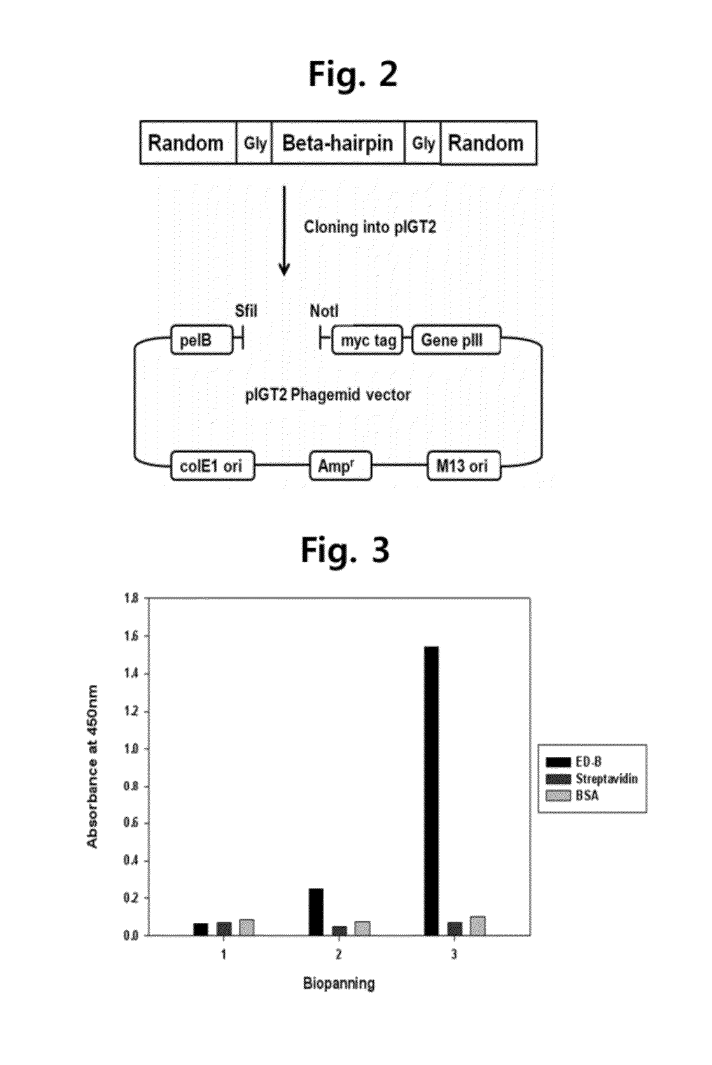

Double strand DNA was prepared by PCR reaction using two degenerate oligonucleotides and restricted with restriction enzymes, SfiI and NotI. Then, DNA was cloned into pIGT2 phagemid vector, constructing a library of not less than 8×10⁸ (FIG. 2).

### Example 10

### Biopanning

Biopanning to fibronectin ED-B, VEGF, VCAM1, nAchR or HAS protein was carried out 3-5 times using a bipodal-peptide binder library, and the ratio of output phage to input phage of phage peptides recovered from each biopanning step was determined (Table 1a).

TABLE 1a
Biopanning to fibronectin ED-B protein.

| Panning round (times) | Input phage (pfu) | Output phage (pfu) | calculation |
|---|---|---|---|
| 1 | 2.8 × 10¹¹ | 1.1 × 10⁷ | 4.0 × 10⁻⁵ |
| 2 | 1.6 × 10¹¹ | 1.0 × 10⁷ | 5.1 × 10⁻⁵ |
| 3 | 1.6 × 10¹¹ | 2.1 × 10⁷ | 1.3 × 10⁻⁵ |

TABLE 1b
Biopanning to VEGF protein.

| Panning round (times) | Input phage (pfu) | Output phage (pfu) | calculation |
|---|---|---|---|
| 1 | 1.0 × 10¹¹ | 1.0 × 10⁶ | 10 × 10⁻⁵ |
| 2 | 2.8 × 10¹⁰ | 6.5 × 10⁶ | 23 × 10⁻⁵ |
| 3 | 1.9 × 10¹⁰ | 3.1 × 10⁷ | 189 × 10⁻⁵ |
| 4 | 1.3 × 10¹¹ | 2.1 × 10⁸ | 161 × 10⁻⁵ |
| 5 | 3.5 × 10¹¹ | 3.7 × 10⁷ | 100 × 10⁻⁵ |

TABLE 1c
Biopanning to VCAM1 protein.

| Panning round (times) | Input phage (pfu) | Output phage (pfu) | calculation |
|---|---|---|---|
| 1 | 5.4 × 10¹⁰ | 1.4 × 10⁶ | 2.5 × 10⁻⁵ |
| 2 | 4.1 × 10¹¹ | 2.3 × 10⁶ | 0.5 × 10⁻⁵ |
| 3 | 1.0 × 10¹² | 3.4 × 10⁷ | 3.4 × 10⁻⁵ |
| 4 | 4.0 × 10¹² | 1.5 × 10⁸ | 3.7 × 10⁻⁵ |
| 5 | 7.9 × 10¹⁰ | 3.3 × 10⁶ | 4.1 × 10⁻⁵ |

TABLE 1d
Biopanning to nAchR protein.

| Panning round (times) | Input phage (pfu) | Output phage (pfu) | calculation |
|---|---|---|---|
| 1 | 2.6 × 10¹² | 9.9 × 10⁷ | 3.1 × 10⁻⁵ |
| 2 | 7.9 × 10¹¹ | 4.6 × 10⁷ | 5.8 × 10⁻⁵ |
| 3 | 2.0 × 10¹² | 5.6 × 10⁸ | 28.3 × 10⁻⁵ |
| 4 | 3.3 × 10¹² | 3.2 × 10⁹ | 97.6 × 10⁻⁵ |
| 5 | 3.3 × 10¹¹ | 6.7 × 10⁸ | 202 × 10⁻⁵ |

TABLE 1e
Biopanning to HSA protein.

| Panning round (times) | Input phage (pfu) | Output phage (pfu) | calculation |
|---|---|---|---|
| 1 | 2.6 × 10¹¹ | 1.7 × 10⁷ | 6.5 × 10⁻⁵ |
| 2 | 5.5 × 10⁹ | 5.4 × 10⁶ | 100 × 10⁻⁵ |
| 3 | 4.1 × 10¹⁰ | 3.0 × 10⁷ | 75 × 10⁻⁵ |
| 4 | 1.4 × 10¹⁰ | 5.8 × 10⁷ | 400 × 10⁻⁵ |
| 5 | 2.0 × 10⁹ | 4.0 × 10⁷ | 1,000 × 10⁻⁵ |

TABLE 1f
Biopanning to MyD88 protein.

| Panning round (times) | Input phage (pfu) | Output phage (pfu) | calculation |
|---|---|---|---|
| 1 | 2.0 × 10¹¹ | 2.8 × 10⁷ | 14 × 10⁻⁵ |
| 2 | 1.3 × 10¹¹ | 1.0 × 10⁷ | 7.7 × 10⁻⁵ |
| 3 | 1.1 × 10¹⁰ | 1.8 × 10⁸ | 163 × 10⁻⁵ |
| 4 | 4.0 × 10¹² | 3.3 × 10⁹ | 8.2 × 10⁻⁵ |
| 5 | 7.0 × 10¹⁰ | 1.8 × 10⁸ | 257 × 10⁻⁵ |

### Example 11

### ELISA of Input Phage to Fibronectin ED-B

ELISA of each input phage of bipodal-peptide binder library was carried out for ED-B, streptavidin and BSA. Binding property of first input phages was similar in all ED-B, streptavidin and BSA, whereas the absorbance of ED-B in second input phage was 5.1-fold and 3.4-fold higher than that of streptavidin and BSA, respectively. The binding property of ED-B in third input phage was 22-fold and 15-fold higher than that of streptavidin and BSA, respectively, suggesting that biopanning to ED-B is successful (FIG. 3 and Table 2).

TABLE 2

| Type | Input phage 1 | Input phage 2 | Input phage 3 |
|---|---|---|---|
| ED-B | 0.062 | 0.249 | 1.544 |
| Streptavidin | 0.070 | 0.048 | 0.068 |
| BSA | 0.088 | 0.073 | 0.102 |

### Example 12

### Detection of Phage Peptide Specific to Fibronectin ED-B, VEGF, VCAM1, nAchR, HAS and MyD88 protein (Phage ELISA) and Sequencing

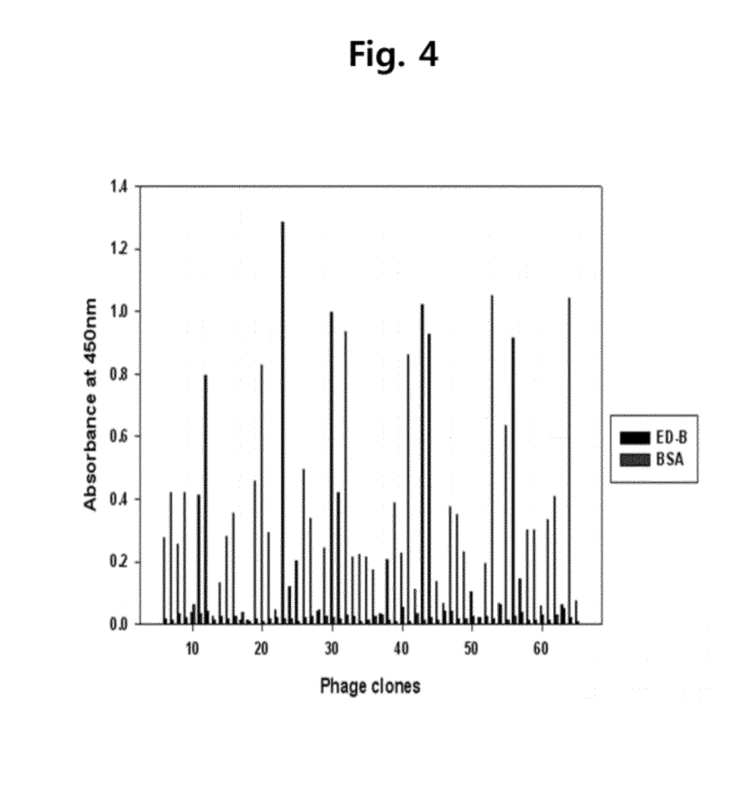

The phages recovered from biopanning step having the highest ratio of output phage to input phage were isolated as plaques. Sixty plaques were amplified from each plaque, and then ELISA for BSA was carried out (FIG. 4). After selecting clones with higher absorbance compared to BSA, they were sequenced. We isolated peptides specific to each protein which were repetitively found in DNA sequencing (Table 3).

TABLE 3a

| | Peptide sequence specific |
|---|---|
| Type | to fibronectin ED-B |
| Peptide 1 | MSADKSGSWTWENGKWTWKGQVRTRD |
| Peptide 2 | HCSSAVGSWTWENGKWTWKGIIRLEQ |
| Peptide 3 | HSQGSPGSWTWENGKWTWKGRYSHRA |

TABLE 3b

| Type | Peptide sequence specific to VEGF |
|---|---|
| Peptide 1 | HANFFQGSWTWENGKWTWKGWKYNQS |
| Peptide 2 | ASPFWAGSWTWENGKWTWKGWVPSNA |
| Peptide 3 | HAFYYTGSWTWENGKWTWKGWPVTTS |
| Peptide 4 | YGAYPWGSWTWENGKWTWKGWRVSRD |
| Peptide 5 | AAPTSFGSWTWENGKWTWKGWQMWHR |

TABLE 3c

| Type | Peptide sequence specific to VCAM1 |
|---|---|
| Peptide 1 | QARDCTGSWTWENGKWTWKGPSICPI |

TABLE 3d

| Type | Peptide sequence specific to nAchR |
|---|---|
| Peptide 1 | EASFWLGSWTWENGKWTWKGKGTLNR |
| Peptide 2 | YAYPLLGSWTWENGKWTWKGWYQKWI |
| Peptide 3 | ASLPAWGSWTWENGKWTWKGWSTRTA |

TABLE 3e

| Type | Peptide sequence specific to HSA |
|---|---|
| Peptide 1 | AASPYKGSWTWENGKWTWKGGWRMKM |
| Peptide 2 | SANSLYGSWTWENGKWTWKGTSRQRW |
| Peptide 3 | YAHVYYGSWTWENGKWTWKGHRVTQT |
| Peptide 4 | YGAYPWGSWTWENGKWTWKGWRVSRD |
| Peptide 5 | YAHFGWGSWTWENGKWTWKGTTDSQS |

TABLE 3f

| Type | Peptide sequence specific to MyD88 |
|---|---|
| Peptide 1 | HSHAFYGSWTWENGKWTWKGNPGWWT |
| Peptide 2 | ASTINFGSWTWENGKWTWKGYTRRWN |

### Example 13

### Affinity Measurement to Fibronectin ED-B, VEGF, VCAM1, nAchR and HAS

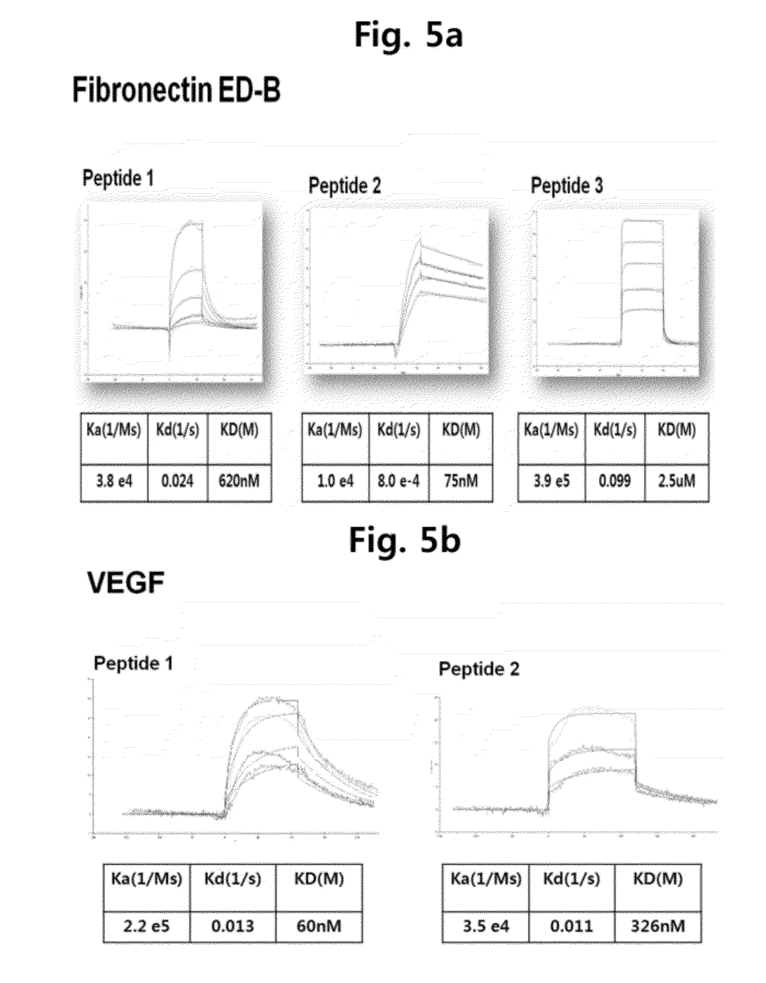

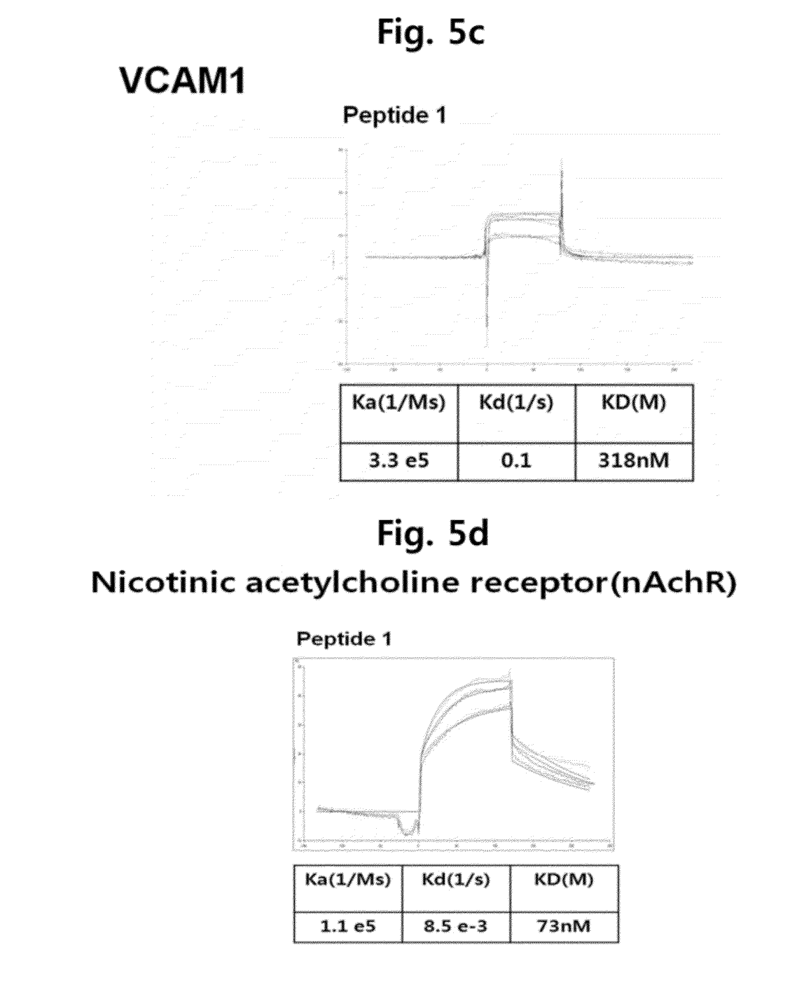

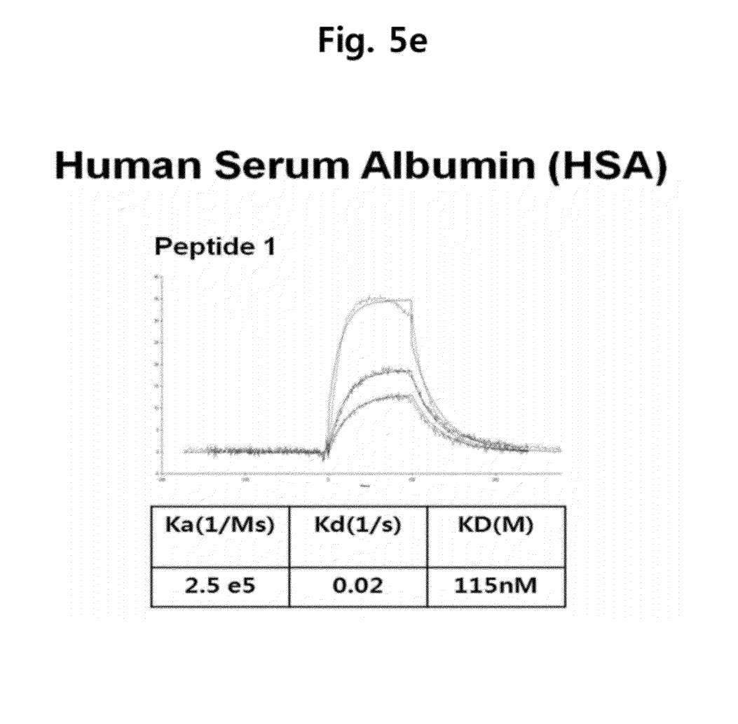

The above-mentioned peptides were synthesized and their affinities to fibronectin ED-B, VEGF, VCAM1, nAchR and HAS were measured using SPR Biacore system (Biacore AB, Uppsala, Sweden). In affinity measurement for fibronectin ED-B, each peptide 1, 2 and 3 was 620 nM, 75 nM and 2.5 μM (FIG. 5a). In VEGF, peptide 1 and 2 exhibited an affinity of 60 nM and 326 nM (FIG. 5b), respectively. In peptide fragment for VCAM1, peptide 1 had an affinity of 318 nM (FIG. 5c). In peptide fragment for nAchR, peptide 1 had an affinity of 73 nM (FIG. 5d). Finally, peptide 1 was 115 nM in affinity measurement to peptide fragment for HSA (FIG. 5e).

### Example 14: Specificity Analysis to Fibronectin ED-B, VEGF, VCAM1, nAchR and HAS

Specificity of recombinant phages to each protein was carried out using ELISA.

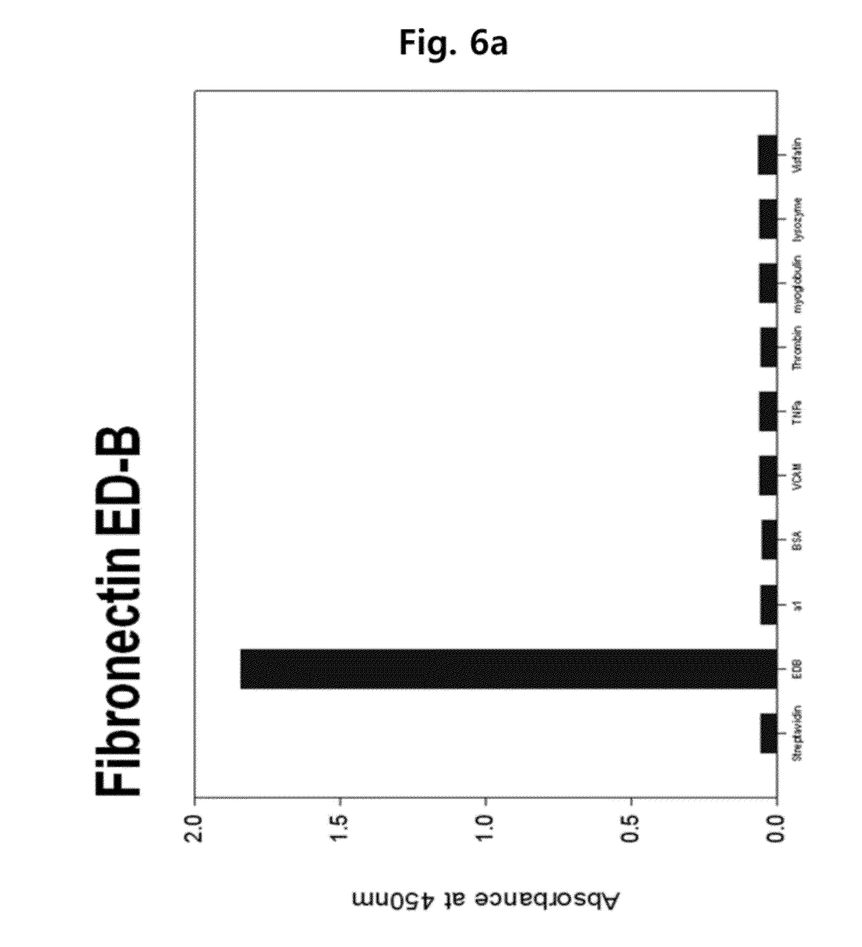

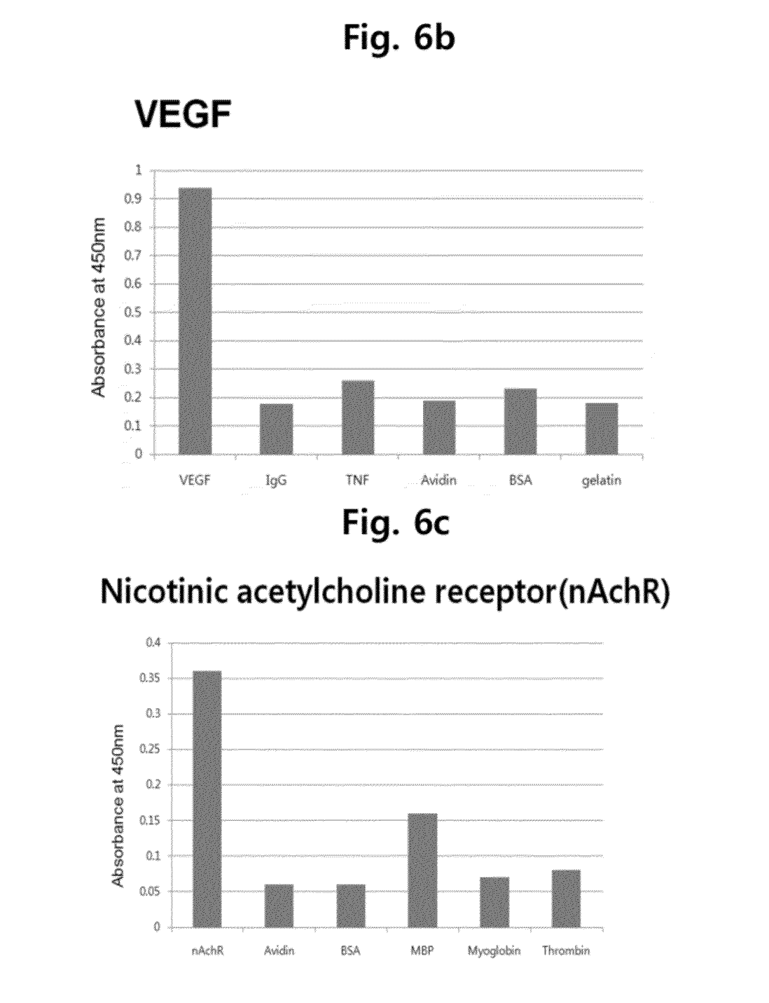

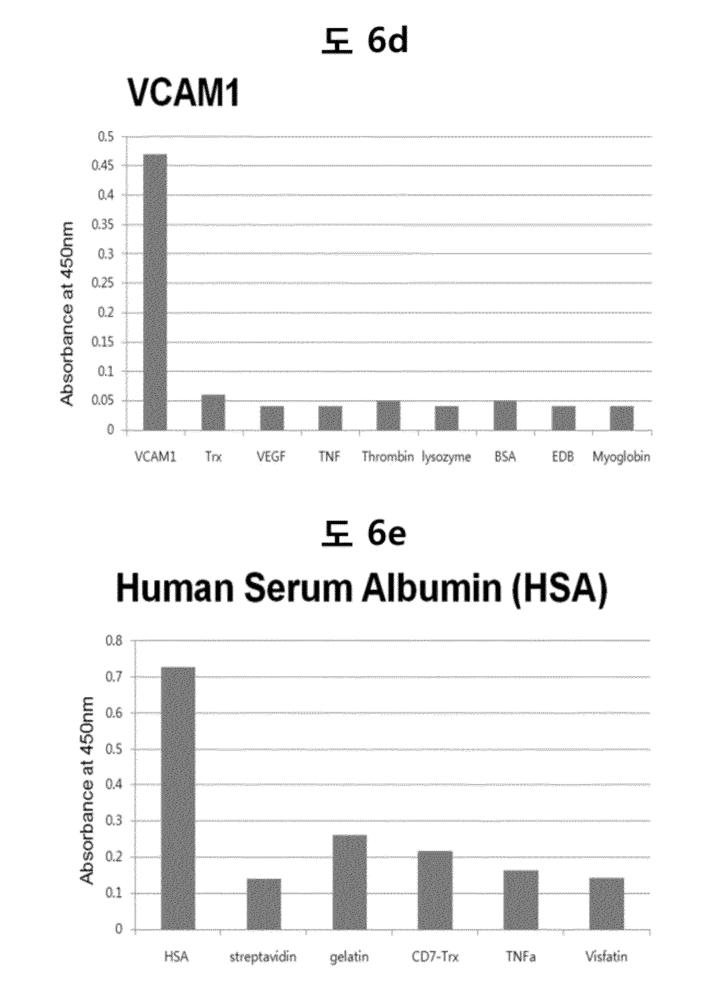

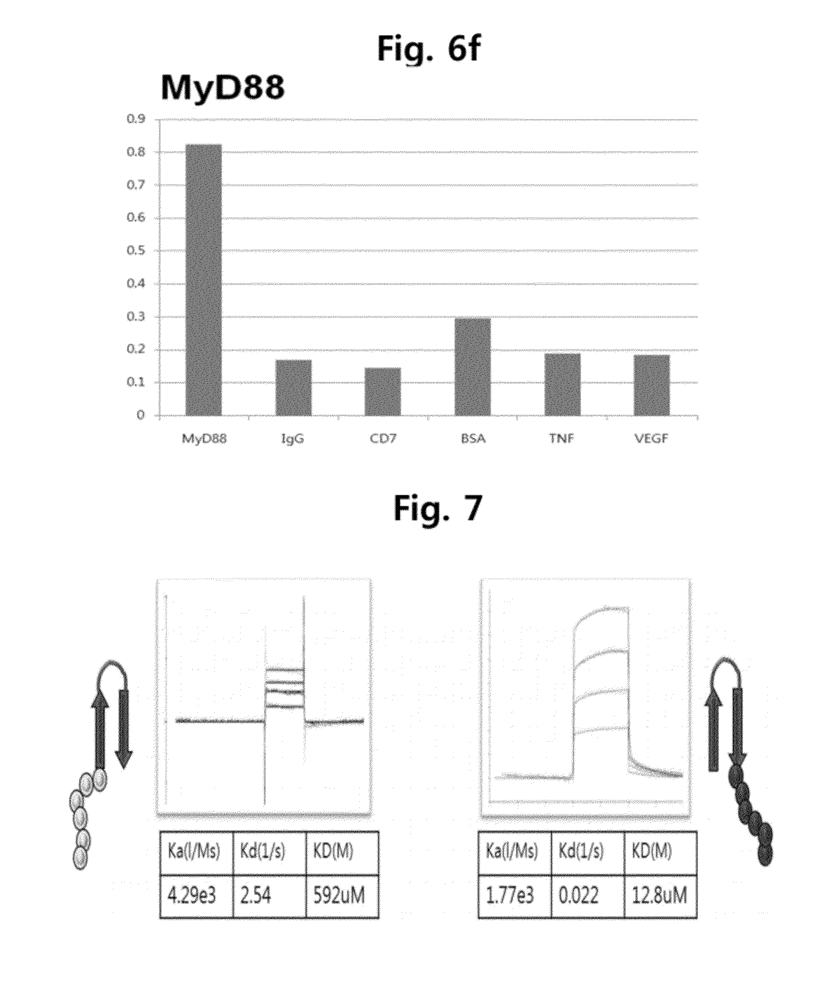

Each protein (5 μg/ml) was seeded into wells (50 μl per well) in a 96-well ELISA plate and next day, all wells were washed with 0.1% PBST (Tween-20) three times, and blocked at room temperature for 2 hrs using 2% skim milk. Then, the solution was completely removed and the plate was washed with 0.1% PBST three times. Recombinant phages containing the peptide of the present invention were thoroughly mixed with 2% BSA. Each mixture (100 μl) was divided into wells coated with 10 proteins and kept to stand at 27° C. for 2 hrs. After washing with 0.1% PBST 5 times, HRP-conjugated anti-M13 antibodies (1:1,000 dilution; GE Healthcare) were added to each well and incubated at 27° C. for 1 hr. After washing with 0.1% PBST 5 times, 100 μl TMB was divided into each well to induce colorimetric reaction, followed by stopping the reaction adding 100 μl of 1 M HCl. The absorbance was measured at 450 nm. As shown in FIG. 6a, the absorbance of peptide 2 (Table 3a) specific to ED-B isolated from bipodal-peptide binder was measured above 30-fold higher than that of other proteins, suggesting that peptide 2 sequence is specific to ED-B. As shown in FIGS. 6b-6f, it could be appreciated that each peptide 1 in Table 3b-3f has specificity for VEGF, VCAM1, nAchR, HSA and MyD88.

### Example 15

### Cooperative Effect of SPR (Surface Plasmon Resonance)

To verify cooperative effect of bipodal-peptide binder to antigen, we synthesized two peptides removing either N- or C-terminal region of peptide 2 to ED-B having excellent specificity in Table 3a for affinity measurement. Affinity of N-terminal region and C-terminal region was measured at 592 μM and 12.8 μM, respectively (FIG. 7). It was demonstrated that cooperative effect is generated by bipodal structure necessary in bipodal-peptide binder, and measured at an affinity of 43 nM (FIG. 5a).

### Example 16

### Binding Assay to Other β-Hairpin

In addition to tryptophan zipper, GB1m3 and HP7 peptide as a type of other β-hairpin backbones were synthesized to contain N-terminal sequence (HCSSAV) and C-terminal sequence (IIRLEQ) of peptide 2 which is specifically bound to ED-B (Anigen, Korea). In other words, the sequence of bipodal-peptide binder in tryptophan zipper is HCSSAVGSWTWENGKWTWKGIIRLEQ, and in GB1m3 and HP7 are HCSSAVGKKWTYNPATGKFTVQEGIIRLEQ and HCSSAVGKTWNPATGKWTEGIIRLEQ, respectively. Affinity of each peptide was measured using BIAcore X (Biacore AB,

Uppsala, Sweden). ED-B was immobilized on streptavidin (SA) chip (Biacore) by injecting 2,000 RU biotinylated-EDB. PBS (pH 7.4) was used as a running buffer.

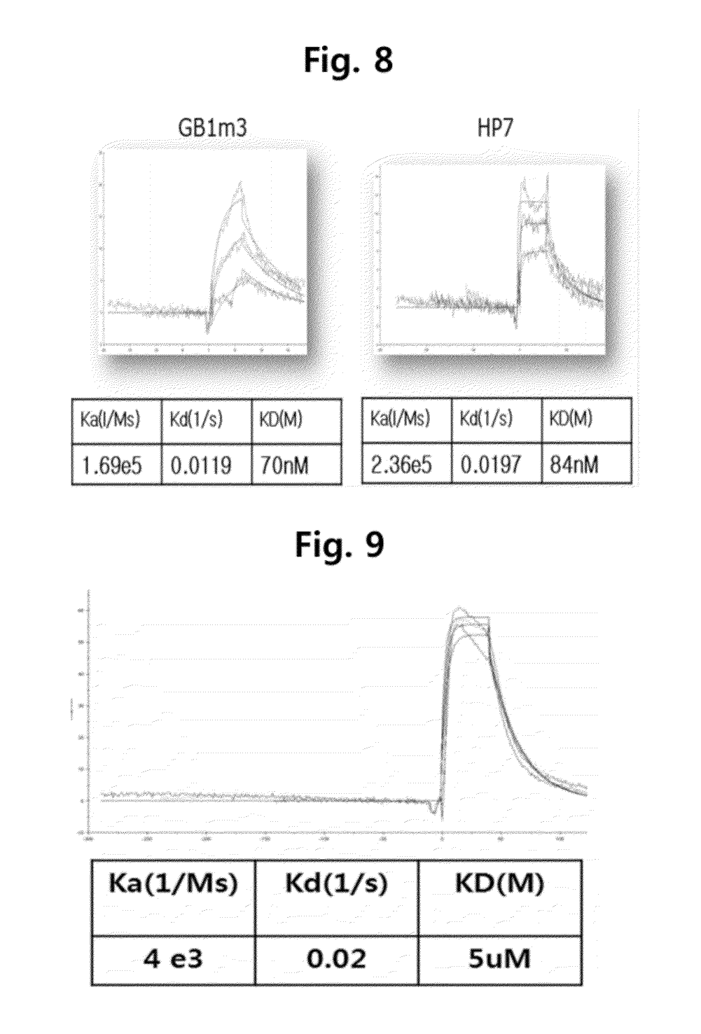

Kinetics at different concentrations was measured under a flow rate of 30 μl/min, and affinity was calculated using BIAevaluation software. As a result, affinity of each GB1m3 and HP7 was 70 nM and 84 nM, demonstrating that affinities of both GB1m3 and HP7 are similar to that of tryptophan zipper (43 nM) (FIG. 8). It could be appreciated that all stable β-hairpin motifs may function as a structure stabilizing region.

### Example 17

### Binding Assay to Bipodal-peptide Binder Containing Leucine Zipper as a Structure Stabilizing Region

A leucine zipper motif as a structure stabilizing region instead of β-hairpin structure was synthesized to contain N-terminal sequence (HCSSAV) and C-terminal sequence (IIRLEQ) of peptide 2 which is specifically bound to ED-B, producing two peptides, CSSPIQGGSMKQLEDWEELLSKNYHLENEVARLKKLVGER and IIRLEQGGSMKQLEDKVEELLSKNYHLENEVARLKKLVGER (Anigen, Korea). Both peptides were formed as dimer, and their affinities were measured using BIAcore X (Biacore AB,

Uppsala, Sweden). As a result, affinity of leucine zipper was 5 μM, demonstrating that affinities of leucine zipper are lower than that of tryptophan zipper (43 nM). However, it may be possible to utilize a leucine zipper as a structure stabilizing region in bipodal-peptide binder (FIG. 9).

### Example 18

### Cancer Targeting of Bipodal-peptide Binder Specific to Fibronectin ED-B as a Cancer Biomarker

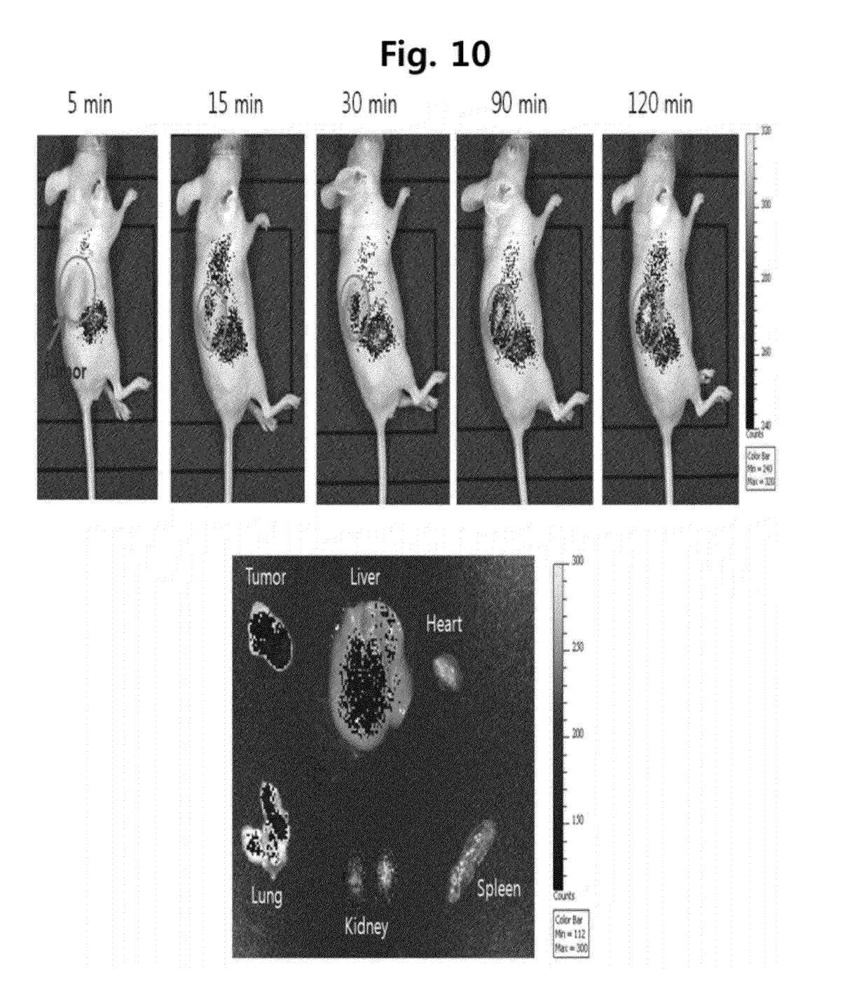

After Cy5.5-NHS fluorescence dye was linked to bipodal-peptide binder which targets fibronectin ED-B widely distributed in cancer cells, mice injected with human U87MG cells were intravenously administered with bipodal-peptide binder-Cy5.5, followed by measuring fluorescence through IVIS to determine whether the bipodal-peptide binder may target cancerous tissue (FIG. 10). As a result, it was shown that the bipodal-peptide binder specific to fibronectin ED-B as a cancer biomarker was accumulated in cancer tissue, suggesting that the bipodal-peptide binder of the present invention may be efficiently utilized in in vivo imaging.

### Example 19

### GST-BPB Fusion Protein for Fibronectin EDB

### 1. Preparation of GST-BPB Gene

The BPB sequence recognizing fibronectin EDB was cloned into pGEX4T-1 vector (GE Healthcare) containing the glutathione S-transferase (GST) gene. Using two oligonucleotides, GST-F1 (5′-ACCGGATCCCATTGTTCTAGT-3′) and GST-B1 (5′-ATTCTCGAGTTACGCTCCTCCTCC-3′), a nucleotide sequence of the BPB capable of binding to fibronectin EDB was obtained by PCR using the phagemid vector as templates (Examples 1 and 3) obtained from phages bound to the EDB protein. The amino acid sequence of the BPB obtained is: HCSSAVGSWTWENGKWTWKGIIRLEQ. Twenty pmol GST-F1, 20 μmol GST-B1, 1 μl the phagemid template 4 μl dNTP mixture (2.5 mM), 1 μl ExTaq DNA polymerase (10 U) and 5 μl 10×PCR buffer were mixed and then distilled water was added to a final volume of 50 μl, preparing the mixture solution. The BPB insert was prepared by performing PCR (5 min at 94° C.; 30 cycles-30 sec at 94° C.; 30 sec at 55° C.; and 1 min at 72° C.) and purified using a PCR purification kit (GeneAll, Seoul, Korea). To clone the insert into the pGEX4T-1 vector, the BPB insert and the pGEX4T-1 vector were digested with restriction enzymes. About 2 μg BPB insert were digested with BamHI (NEB) and Xhol (NEB) for 4 hrs and purified using a PCR purification kit. The vector and the insert were mixed at a molar ratio of 1:3 (vector to insert) and ligated at 18° C. for 10 hrs using T4 DNA ligase (Bioneer). After transformation to E. coil XL-1 competent cells (Stratagene), the transformed cells were spread in agar media containing ampicillin. The colony grown on a solid agar plate was inoculated into 5 ml LB media, and then incubated at 37° C. overnight with shaking at 200 rpm. Plasmids were purified by a plasmid preparation kit (GeneAll, Seoul, Korea), and then sequenced to determine whether the cloning is successful.

### 2. Expression and Purification of GST-BPB Gene

After transformation of the vector carrying the GST-BPB gene into E coli BL21 cells, the transformed cells were inoculated into 2 liter of LB containing ampicillin (25 μg/ml) and then cultured to OD=0.6-0.8. Afterwards, 1 mM isopropyl-β-D-thiogalactopyranoside (IPTG) were added to the media and cultured at 37° C. for 8 hrs with shaking at 200 rpm. After removing the supernatant through centrifugation at 4,000×g for 20 min, the precipitated cells were resuspended in a lysis buffer (50 mM sodium phosphate (pH 8.0), 300 mM NaCl and 5 mM imidazole). After storing at −80° C. overnight, the E coli cells were lysed using a sonicator and then centrifuged at 15,000×g for 1 hr. The supernatant was applied to GST affinity resin (Peptron), followed by washing the resin with PBS. The GST-BPB protein was collected using an elution buffer (containing 20 mM glutathione in 10 mM Tris-HCl, pH 8). The collected protein was subjected to a gel filtration using Superdex75 column (Amersham) and PBS buffer (pH 7.4) to yield a high-purity GST-BPB protein.

### 3. Evaluation of Binding of the GST-BPB to Fibronectin EDB

We tested using a SPR device whether the purified GST-BPB protein can bind to fibronectin EDB. The affinity measurement was conducted on the Biacore X. The biotin-EDB was fixed on a streptavidin SA CM5 chip (Biacore) up to 2000 RU. PBS (pH 7.4) was used as a running buffer and the flow rate was 30 μ1/min. The kinetics was measured in various concentrations and then the affinity was calculated using BIAevaluation.

### 4. Results

### (1) Preparation of GST-BPB

The BPB recognizing specifically fibronectin ED-B was cloned into the GST fusion vector and expressed and purified in E. coli cells (FIG. 11a).

(2) Affinity measurement of GST-BPB

The affinity of the GST-BPB to fibronectin ED-B was measured using the SPR biacore system. The GST-BPB was analyzed to have the affinity of 284 nM even though the BPB is fused with the GST protein (FIG. 11b).

### Example 20

### TNFα-BPB Fusion Protein

### 1. Preparation of TNFα-BPB Gene

The BPB sequence recognizing either human TNFα (tumor necrosis factor α) or fibronectin EDB was cloned into the pET28b vector (Novagen). Using two oligonucleotides, TNF-F1 (AATAAAACATATGTCTCGAACCCCGA) and TNF-B1 (ATGGATCCCAGGGCAATGATC), the TNFα gene was amplified using the Gh75 vector carrying the human TNFα gene (Cytokine Bank, Korea) as templates and cloned into the pET28b vector (Novagen). In addition, using two oligonucleotides, BPB-F1 (MT GAATTC TCTTCCTCATCGGGTTCTTCCTCATCGGGTTGTAGTTCTCCTATTC) and BPB-B1 (MT AAGCTT TCA TTGCTCCAACCTAAT), a nucleotide sequence of the BPB capable of binding to fibronectin EDB was obtained by PCR using the phagemid vector as templates (Examples 1 and 3) obtained from phages bound to the EDB protein. The amino acid sequence of the BPB obtained is: CSSPIQGSWTWENGKWTWKGIIRLEQ.

The BPB sequene was cloned into the pET28b vector carrying the TNFα gene. Plasmids were purified by a plasmid preparation kit and then sequenced to determine whether the cloning is successful.

### 2. Expression and Purification of TNFα-BPB

After transformation of the pET28b vector carrying the TNFα-BPB gene into BL21 cells, the transformed cells were plated on an kanamycine agar media. Afterwards, the transformed cells were inoculated into 2 liter of LB containing kanamycine (25 μg/ml) and then cultured to OD=0.6-0.8. Afterwards, 1 mM isopropyl-β-D-thiogalactopyranoside (IPTG) were added to the media and cultured at 37° C. for 8 hrs with shaking at 200 rpm. After removing the supernatant through centrifugation at 4,000×g for 20 min, the precipitated cells were resuspended in a lysis buffer (50 mM sodium phosphate (pH 8.0), 300 mM NaCl and 5 mM imidazole). After storing at −80° C. overnight, the E co/icells were lysed using a sonicator and then centrifuged at 15,000×g for 1 hr. The supernatant was applied to Ni-NTA affinity resin (ELPIS biotech.), followed by washing the resin with the lysis buffer. The TNFα-BPB protein was collected using an elution buffer (containing 50 mM sodium phosphate (pH 8.0), 300 mM NaCl and 300 mM imidazole). The collected protein was subjected to a gel filtration using Superdex75 column and PBS buffer (pH 7.4) to yield a high-purity TNFα-BPB protein.

3. Evaluation of binding of the TNFα-BPB to fibronectin EDB

We tested using a SPR device whether the purified TNFα-BPB protein can bind to fibronectin EDB. The affinity measurement was conducted on the Biacore X. The biotin-EDB was fixed on a streptavidin SA CM5 chip (Biacore) up to 2000 RU. PBS (pH 7.4) was used as a running buffer and the flow rate was 30 μl/min. The kinetics was measured in various concentrations and then the affinity was calculated using BIAevaluation.

### 4. Evaluation of Cytotoxicity of TNFα-BPB

The cytotoxicity of either the TNFα-BPB protein or TNFα was evaluated by MTT assay using L-M mouse fibroblasts. 5000 cells of L-M mouse fibroblasts were cultured for 18 hr on a 96 well microplate and incubated with various concentration of either the TNFα-BPB protein or TNFα for 30 hr, followed by perfoming MTT assay.

### 5. Results

### (1) Preparation of TNFα-BPB

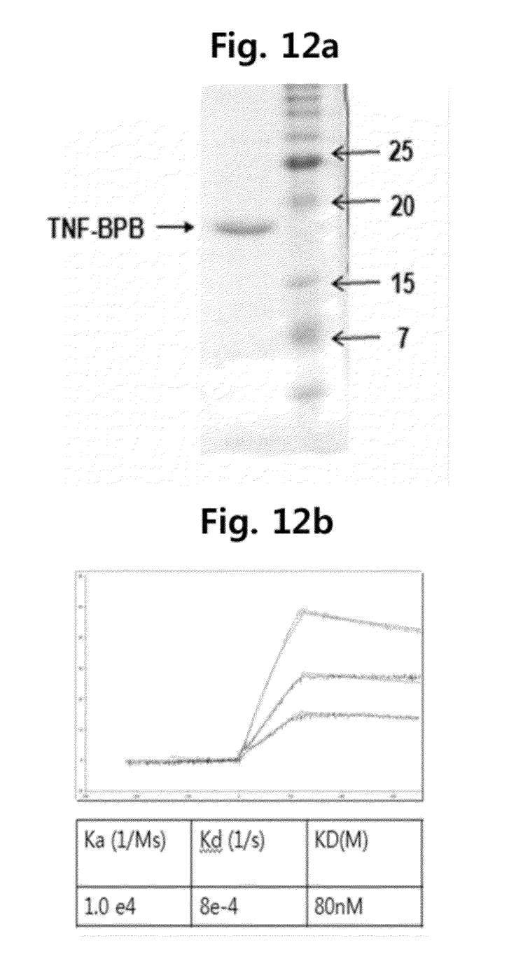

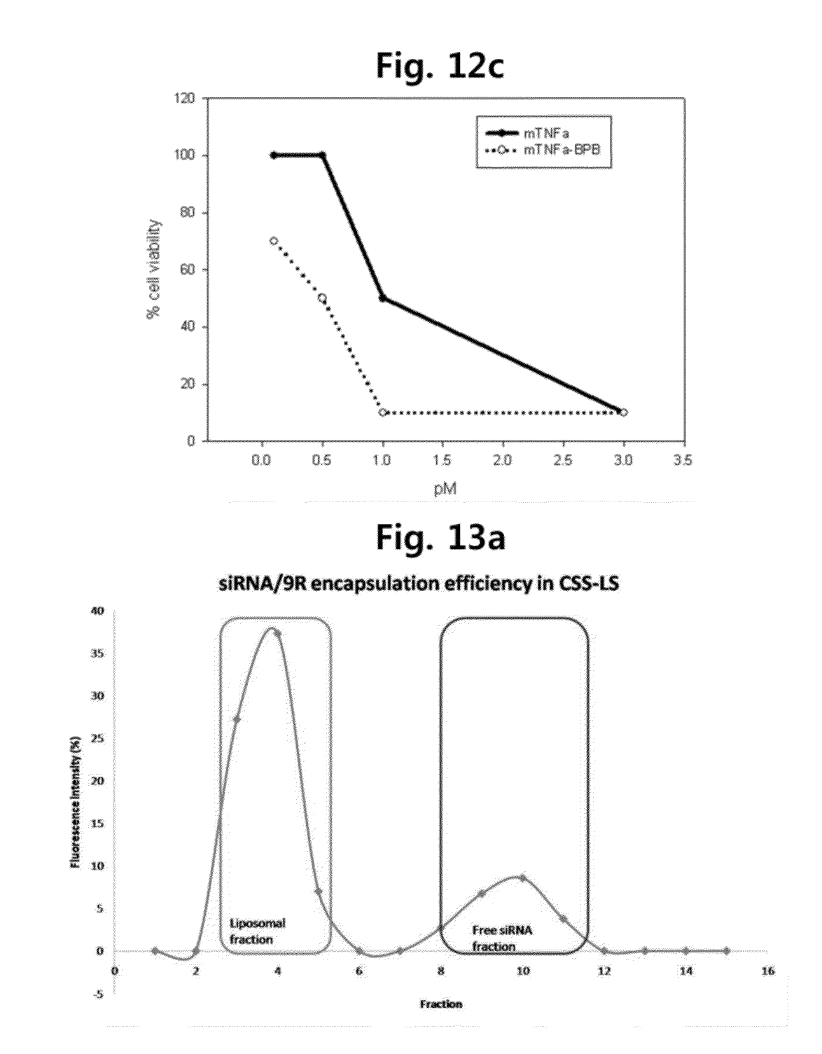

The BPB recognizing specifically fibronectin ED-B was cloned into the vector carrying the TNFα gene and expressed and purified in E. coli cells (FIG. 12a).

### (2) Affinity Measurement of TNFα-BPB

The affinity of the TNFα-BPB to fibronectin ED-B was measured using the SPR biacore system. The GST-BPB was analyzed to have the affinity of 284 nM even though 10 the BPB is fused with the TNFα protein (FIG. 12b).

### (3) Cytotoxicity of TNFα-BPB

The cytotoxicity of either the TNFα-BPB protein or TNFα in L-M mouse fibroblast cells was evaluated by MTT assay. The TNFα-BPB protein has 5-fold higher 15 cytotoxicity than the TNFα protein (FIG. 12c).

### Example 21

### Liposome-BPB

### 1. Conjugation of BPB_{css} to Mal-PEG₂₀₀₀-DSPE

Mal-PEG₂₀₀₀-DSPE was conjugated to the cys residue of the BPB_{EDB} [CSSPIQGSWTWENGK(Cys, a cys residue linked to a lysine residue)WTWKGIIRLEQ]. Briefly, Mal-PEG₂₀₀₀-DSPE (1.1 fold molar excess) was mixed with the BPB_{EDB} in chloroform/DMSO(1:1 v/v) and agitated for 12 hr at room temperature for conjugation.

### 2. Encapsulation Efficiency of 9R/siRNA in BPB_{css}-LS

9R peptide and VEGF-C siRNA (sense 5′ CAG AUG GAU UCC AUG ACA dTdT, anti-sense 5′ AUG UCA UGG MU CCA UGU G dTdT) were diluted to the final volume of 250 μlin HEPES-buffered 5% glucose (pH 7.4) and incubated for 10 min at room temperature. The solution was mixed and voltexed to yield 500 μl of 9R/siRNA complexes at a +/− charge ratio of 1. Using POPC/Chol/POPG/PEG2000-DSPE/BPB-DSPE (4:3:3:1:0.5) mixture for a BPB-DSPE-encapsulating liposome and POPC/Chol/POPG/PEG2000-DSPE (4:3:3:1) mixture for a control liposome, a lipid film was prepared. 500 μl of Hepes buffer glucose 5% (HBG 5%) and 500 μl of 9R/siRNA complex were added to the lipid film. After sonication, extrusion was conducted 11 times using a hand-held extruder (Avanti Polar Lipid, AL, USA) through two polycarbonate membranes (100 nm pore size) stacks. 9R/siRNA-loaded BPB-liposomes (BPB_{css}-LS) and 9R/siRNA-loaded-liposomes as controls were purified by size-exclusion chromatography (CL-4B column). The aliquots of Sepharose CL4B column eluent were analyzed using OliGreen (Invitrogen) to measure contents of siRNA.

### 3. Size Distribution and Zeta-Potentials

The BPB_{css}-LS loaded with 9R/siRNA were diluted with HBS (Hepes Buffer Saline) to obtain an optimal scattering intensity. Hydrodynamic diameter and zeta potential were measured by electrophoretic light scattering using an ELS 8000 apparatus (Otsuka Electronics Korea, Seoul, South Korea).

### 4. Uptake of BPB_{css}-LS in EDB Positive and EDB Negative Cell Line

Cells were grown on cover glass coated with 2% gelatin overnight. Cells were all treated with 200 μg of BPB-liposome (containing 0.3% rhodamine) and liposome as controls (containing 0.3% rhodamine), respectively. After 1 hour treatment at 37° C. for 1 hour, cells were washed and fixed with 4% paraformaldehyde and viewed under confocal microscopy.

5. siRNA Transfection Efficiency in MCF-7 Cells in Vitro

To elucidate the transfection efficiency of siRNA encapsulated in BPB_{css}-LS, MCF-7 cells were transfected with 50 nM VEGF-C siRNA in lipofectamine (positive control), 50 nM BPB_{css}-LS, and 100 nM BPB_{css}-LS, respectively. After 4 hour transfection, cells were further incubated for 24 hours before RNA isolation, cDNA synthesis and real-time RT-PCR.

### 6. Results

### (1) Size and Zetα-Potential of BPB-Liposome

The measured size and zetα-potential of BPB_{css}-LS are summarized in the following Table:

TABLE 4

| Size (nm) | S.D. (±) | Zeta potential (mV) | S.D. (±) |
|---|---|---|---|
| 107 | 8.28 | −28.9 | 1.22 |

(2) Encapsulation of siRNA into BPB-Liposome

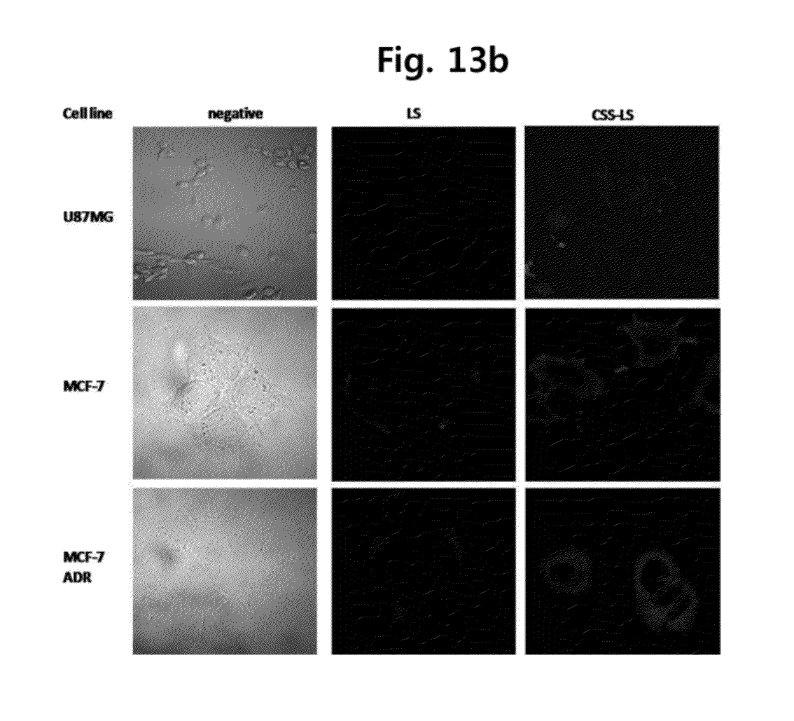

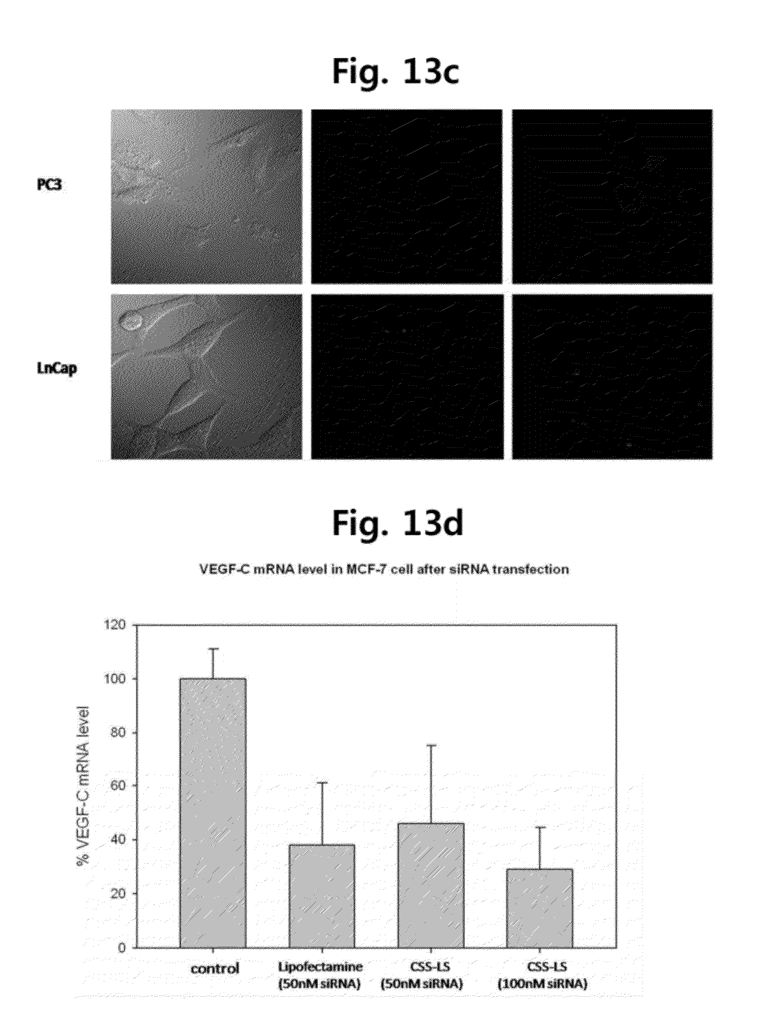

As shown in FIG. 13a, the isolation of the siRNA-encapsulating BPB-lipsome (CSS-LS) and the removal of free siRNA were conducted using the size exclusion CL-4B column. The siRNA/BPB-lipsome with larger size was first obtained and then free siRNA was obtained. As result, the siRNA-encapsulating BPB-lipsome was purified.

### (3) Specificity of BPB-Liposome

The BPB-liposome was tested to recognize EDB as target proteins using EDB-overexpressing U87MG, MCF-7 and MCF-7/ADR cell lines. The three cell lines were incubated with 0.3% rhodamine-containing BPB-liposome or liposome for 1 hr and washed and viewed under microscope. As represented in FIG. 13b, the BPB-liposome (CSS-LS) was well bound to all cell lines. However, liposome (LS) without BPB was not bound to the EDB-overexpressing cell lines. Therefore, it would be appreciated that BPB-liposome can recognize EDB specifically.

Furthermore, the BPB-liposome and the liposome as controls were shown not to be bound to EDB negative cells such as PC3 and LnCap, as represented in FIG. 13c. These results address that the BPB-liposome is significantly specific to EDB.

### (4) Effects of BPB-LS/siRNA on MCF-7 Cells

We tested using MCF-7 cells whether the BPB-LS encapsulating VEGF-C siRNA can suppress mRNA expression of VEGF-C in cells (FIG. 13d). Similarly to lipofectamine conventionally used, the BPB-LS encapsulating either 50 nM VEGF-C siRNA or 100 nM VEGF-C siRNA was analyzed to inhibit mRNA expression of VEGF-C in cells.

### Example 22

### Oleic SPION-BPB

### 1. Preparation of Oleic SPION-BPB

500 μg/100 μl(CHCl₃) of DSPE-PEG₂₀₀₀-Mal (Avanti Polar Lipids, Alabaster, AL, USA) (1,2-Distearoyl-sn-Glycero-3-Phosphoethanolamine-N-[Maleimide(Polyethylene Glycol)2000]) and 250 μg/100 μl(DMSO) of BPB (SSS) peptide (SSSPIOGSWTWENGK(Cys)WTWKGIIRLEO) were reacted overnight in CHCl₃/DMSO co-solven. The occurrence of the reaction was confirmed using MALDI-TOF-MS (SHIMADZU Aximα-CFR, SHIMADZE, Japen). DSPE-PEG₂₀₀₀ polymer, BPB conjugated DSPE-PEG₂₀₀₀ and Rhodamine-DSPE (5:94:1) were mixed in D.W. The Oleic SPION (superparamagnetic iron oxide nanoparticle, synthesized by thermolysis) in hexane was introduced into the polymer solution, sonicated usig a probe-typed sonicator, stirred, centrifuged and finally purified using magnet. The group without BPB was prepared in the same manner as the above.

2. Size Measurement and Analysis of Oleic SPION Coated with BPB-Conjugated DSPE-PEG₂₀₀₀ by ELS and TEM

The hydrodynamic size was measured by electrophoretic light scattering (ELS 8000 instrument of Otsuka Electronics, Otsuka Electronics Korea, Seoul, South Korea) to elucidate BPB conjugation and size change. The stability in D.W. for a period of time was also tested. The aggregation of nanoparticles was examined using transmission electron microscopy (Philips TECNAI F20 instrument (Philips ElectronicInstruments Corp., Mahwah, N.J.)).

3. MR Imaging Evaluation of Oleic SPION Coated with BPB-Conjugated DSPE-PEG₂₀₀₀ by T2-Weighted MR Phantom Study

BPB-conjugated DSPE-PEG₂₀₀₀ coated oleic SPION and DSPE-PEG₂₀₀₀ coated oleic SPION each was added to e-tube and the T2 Time was measured using 3T MRI scanner (Magnetom Avanto, Siemens, Germany), followed by calculation of r2 values.

4. Evaluation of Active Targeting using Cells

U87MG cells were cultured to 90-100% confluency on a 6 well plate in 37° C. CO₂ incubator and incubated with the same concentration of either BPB conjugated DSGPE-PEG₂₀₀₀ coated SPION or DSGPE-PEG₂₀₀₀ coated SPION for 30 min. After cell harvest, the signal intensity was measured using 3T MRI scanner (Magnetom Avanto, Siemens, Germany). In addition, rhodamine-derived signals in cells were observed under a confocal microscope (Olympus, FV1000).

### 5. Cytotoxicity Evaluation

U87MG cells were cultured to 50% confluency on a 96 well plate in 37° C. CO₂ incubator and incubated with the same concentration of either BPB conjugated DSGPE-PEG₂₀₀₀ coated SPION or DSGPE-PEG₂₀₀₀ coated SPION for 12 hr. The MTT assay was carried out for evaluating cytotoxicity.

6. Active Targeting MR Experiment using Brain Cancer Animal Model

5×10⁶ U87MG cells were implanted into the right rear leg of Balb/c nude(female) 6-week aged mice and grown for 2 weeks to the tumor size of about 100-150 mm³. 20 mg Fe/kg of BPB-conjugated DSPE-PEG₂₀₀₀ coated oleic SPION or DSPE-PEG₂₀₀₀ coated oleic SPION was administered to the mice via tail vein, followed by monitoring time-dependent signal intensity in tumor region using 3T MRI.

### 7. Results

### (1) Preparation of SPION-BPB

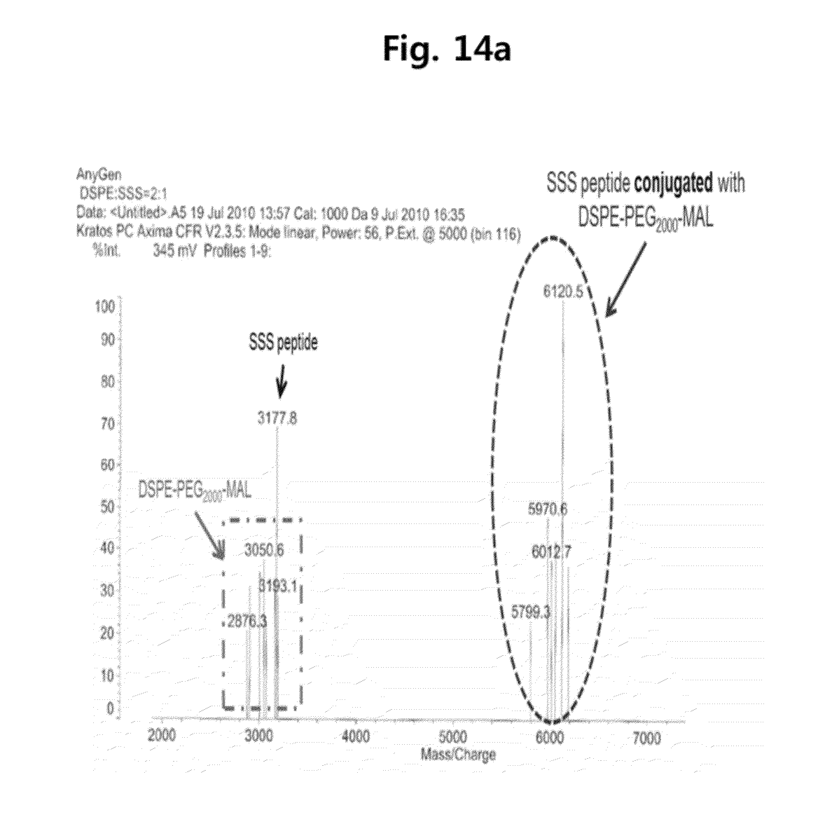

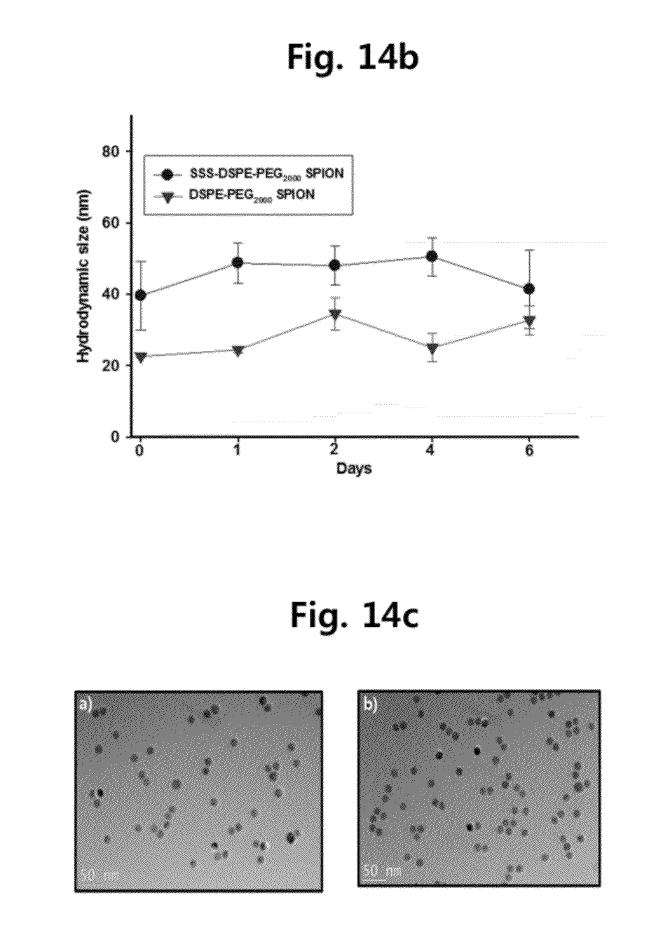

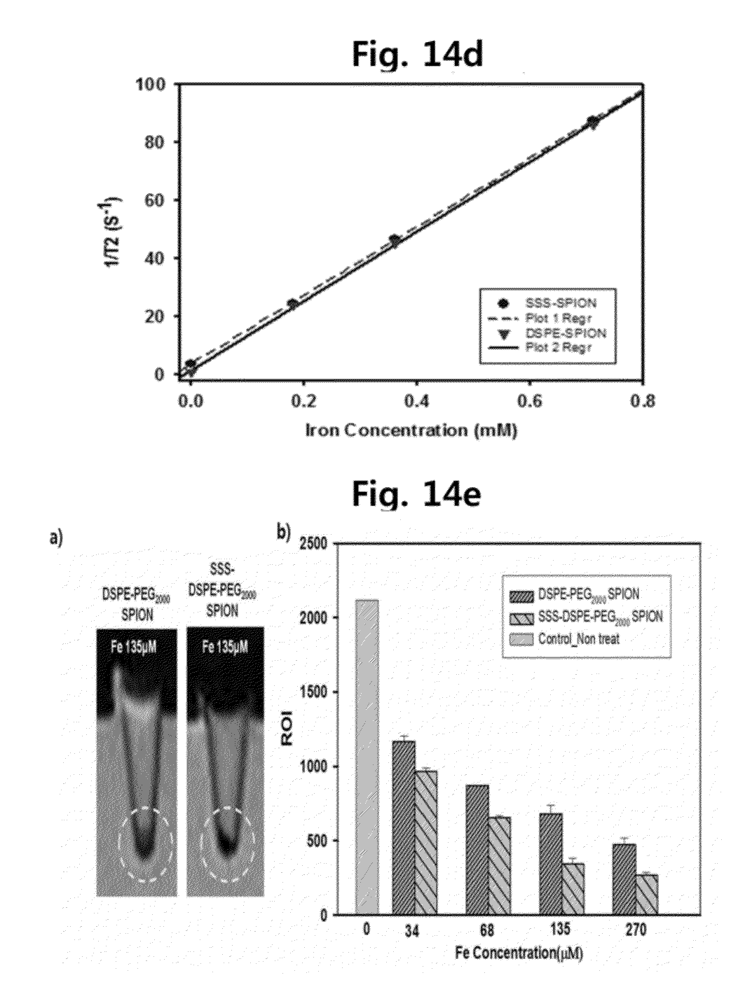

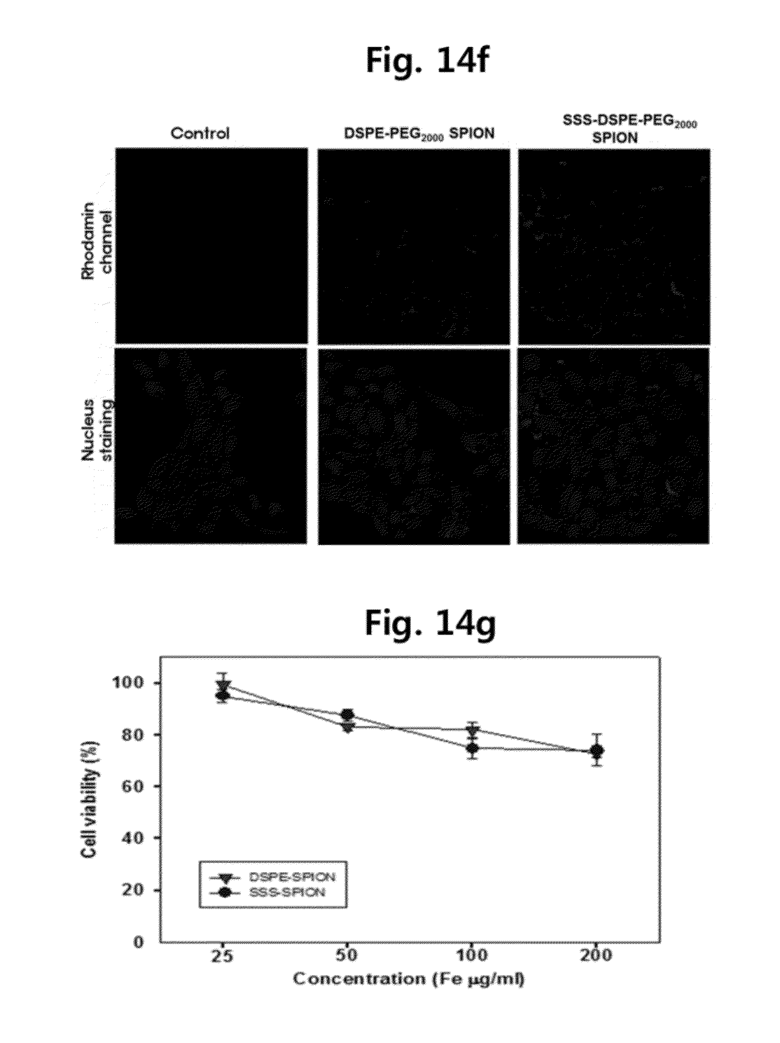

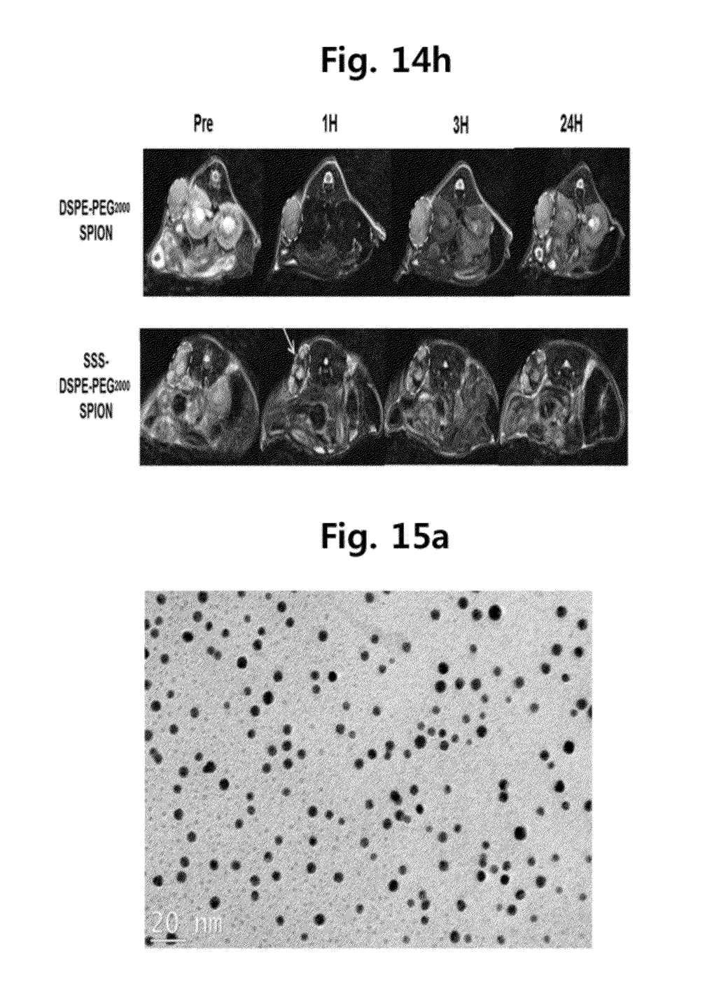

The conjugation of DSPE-PEG₂₀₀₀-Mal and BPB(SSS) peptide was confirmed by MALDI (FIG. 14a).

The hydrodynamic size (size in aqueous solution) of the SPION synthesized using DSPE was measured about 30 nm. The size of the SPION synthesized using DSPE conjugated with BPB (SSS peptide) became larger by about 20 nm.

TABLE 5
Hydrodynamic size measurement

| | Hydrodynamic |
|---|---|
| | Size(nm) |
| DSPE-PEG₂₀₀₀ SPION | 30.6 ± 4.8 |
| SSS-DSPE-PEG₂₀₀₀ SPION | 47.0 ± 2.5 |

The changes in the hydrodynamic size were monitored for one week by ELS. Little or no changes in size were observed, demonstrating that the nanoparticle was stable (FIG. 14b). The TEM images address that the nanoparticle (SPION) conjugated with BPB is well dispered. In addition, the core size was measured about 12 nm (FIG. 14c).

(2) MR Imaging Evaluation of Oleic SPION Coated with BPB-Conjugated DSPE-PEG₂₀₀₀ by T2-Weighted MR Phantom Study

DSPE-PEG₂₀₀₀ coated oleic SPION and BPB conjugated DSPE-PEG₂₀₀₀ coated oleic SPION all were measured to have r2 value of 120 mM⁻¹S⁻¹, addressing that they are useful as MRI T2 imaging agents (FIG. 14d).

(3) Evaluation of Active Targeting using Cells

MR images and signal intensities in the group with BPB and the group without BPB were evaluated (FIG. 14e). MR images for BPB conjugated DSPE-PEG₂₀₀₀ coated SPION were shown to be darker, because the content of the SPION uptaken by cells was larger than that without BPB. In addition, MR signals (ROI signal) for BPB conjugated DSPE-PEG₂₀₀₀ coated SPION were analyzed to be lower than those of DSPE-PEG₂₀₀₀ coated SPION.

Rhodamine-derived signals of _{DSPE-PEG2000} coated SPION and BPB-conjugated DSPE-PEG₂₀₀₀ coated SPION were compared using a confocal microscope (FIG. 14f).

The groups with BPB conjugation was analyzed to show higher rhodamine signal than that without BPB conjugation.

### (4) Cytotoxicity Evaluation

All _{DSPE-PEG2000} coated SPION and BPB conjugated DSPE-PEG₂₀₀₀ coated SPION were analyzed by the MTT assays to have little or no cytotoxicity (FIG. 14g).

(5) Active targeting MR experiment using brain cancer animal model

In the brain cancer anminal model, the tumor region became darker 1 hr after injection of BPB conjugated DSPE-PEG₂₀₀₀ coated SPION (FIG. 14h). In contrast, a negligent change in the tumor region was observed after injection of DSPE-PEG₂₀₀₀ coated SPION with no BPB conjugate.

### Example 23

### Gold NPs-BPB

### 1. Preparation of Pegylated Gold Nanoparticles

Gold nanoparticles with the size of about 5 nm were synthesized using gold ion and reducing agents. The coating of gold nanoparticles with biocompatible polymeric PEG is necessary to reduce uptake by RES (reticuloendothelial system) (i.e., in vivo stability) and to increase delivery efficiency to target cells. To introduce a thiol group into PEG, PEG was reacted with lipoic acid containing a dithiol group and a carboxyl group. The carboxy group of lipoic acid was actived with DCC (dicyclohexylcarbodiimide) and NHS (N-hydroxylsucciniimide) and reacted with 100 μg of NH₂-PEG-OCH₃ to give lipoic-PEG. The coating of gold nanoparticles with PEG was confirmed in 1 M NaCl by aggregation measurement.

2. Gold nanoparticle-BPB Conjugation

The synthesized lipoic-PEG-maleimde and BPB (CSS peptide, CSSPIQGSWTWENGK(cys)WTWKGIIRLEQ) with exposed thiol group were reacted overnight in CHCl₃/DMSO(3:1) co-solvent and coated using lipoic-PEG-maleimde-BPB.

### 3. Evaluation on Cancer Cell Recognition of Gold Nanoparticle-BPB

The recognition of BPB-gold nanoparticles to fibronectin ED-B was analyzed ysing fibronectin ED-B-overexpressing U87MG glioblastoma cells. Human U87MG glioblastoma cells were grown on cover slip to 90-100% confluency and incubated with BPB-gold nanoparticles for 1 hr 37° C. Afterwards, cells were washed three times with PBS and trypsinized, followed by measurement of intracellular Au content using Inductively coupled plasma mass spectrometry (ICP-MS).

4. Coating of Gold Nanoparticles with Zwitterions

While gold nanoparticles may overcome shortcomings of conventional imaging agents (short imaging time, nephrotoxicity), they have problem of poor excretion due to their size. To overcome such problem, the gold nanoparticles were coated with zwitterions not only exhibiting functions of PEG but also significantly increasing hydrodynamic size, produing the final hydrodynamic size of no more than 8 nm for excretion. By introducing a cyclic dithio group into a part of zwitterions binding directly to gold nanoparticles, the stability between gold nanoparticles and zwitterions was increased. The chemical structure of the zwitterion used is represented as follows:

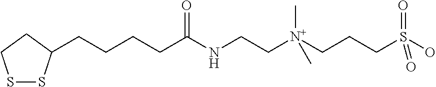

The optimal ratio of gold nanoparticle to zwitterions for stability in serum and 1 M NaCl was determined by measuring peak changes of surface plasmon resonance for gold nanoparticles. Various ratios of gold nanoparticle to zwitterions (1:3000, 1:5000, 1:7000, 1:10000, 1:12000 and 1:15000) were reacted for 12 hr and isolated by centrifugation. After incubation in 10% serum and 1 M NaCl for 24 hr, the peaks of surface plasmon resonance were obtained by UV-Vis spectrum.

### 5. BPB Modification for BPB Conjugation of Gold Nanoparticles

For binding between gold nanoparticles and BPB (i.e., increase of serum stability), a cyclic dithio group was also introduced into BPB. Lipoic acid was incubated in DCC(N,N′-Dicyclohexylcarbodiimide)/NHS(N-hydroxl succinimides) and THF (tetrahydrofuran) for 72 hr to produce lipoic-NHS, followed by purification by recrystallization using toluene. The purified lipoic-NHS was reacted with the amine group (NH₂) of lysine residue of BPB having acetylated N-terminal in DMSO (dimethyl sulfoxide) for 2 hr to introduce the cyclic dithio group into BPB.

### 6. Results

### (1) Preparation of Gold Nanoparticle-BPB

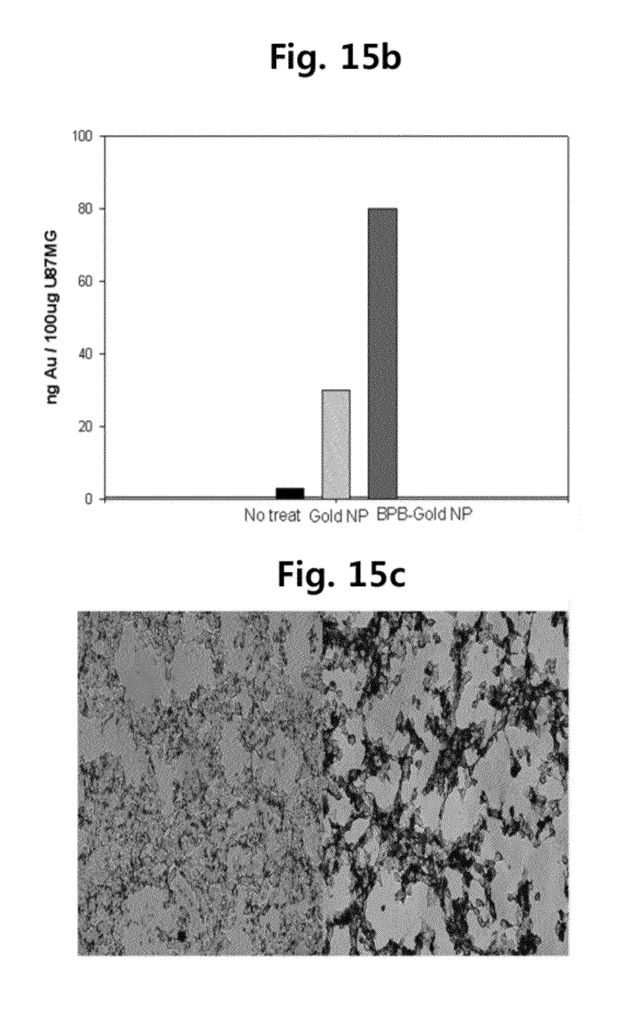

Gold nanoparticles may overcome shortcomings associated with conventional iodine-based CT imaging agents such as short imaging time and nephrotoxicity. Furthermore, gold nanoparticles cotated with biocompatible polymer for imaging liver cancer have been considered next-generation CT imaging agents. We developed gold nanoparticles for CT cancer imaging by conjugation of tumor targeting BPB. Gold nanoparticles with the size of 5 nm were conjugated with BPB recognizing fibronectin EDB. The nanoparticles conjugated with BPB were analyzed by TEM images to show excellent dispersion (FIG. 15a).

### (2) Tumor Targeting of Gold Nanoparticle-BPB

U87MG cells were incubated with BPB-conjugated gold nanoparticles and intacellular Au content was measured by ICP-MS. Cells treated with nanoparticles with BPB were observed to show higher intacellular Au content those treated with nanoparticles without BPB. Therefore, it would be understood that BPB-conjugated gold nanoparticles can be well bound to cell surface via EDB and effectively delivered into cells (FIG. 15b). Compared with gold nanoparticles not conjugated with BPB, BPB-conjugated gold nanoparticles were observed by silver enhancement microscope to be absorbed to U87MG cells in more selective manner (FIG. 15c).

(3) Coating of Gold Nanoparticles with Zwitterions

To increase the serum stability of BPB-conjugated gold nanoparticles, BPB-conjugated gold nanoparticles were surface-coated with zwitterions. Where the ratio of gold nanoparticle to zwitterions is no less than 1:10000, peaks of surface plasmon resonance for gold nanoparticles were not altered 24 hr after incubation in serum or 1 M NaCl. These results demonstrate that gold nanoparticles coated with zwitterions show excellent stability in serum or 1 M NaCl.

### (4) BPB Modification for BPB Conjugation of Gold Nanoparticles

Lipoic-BPB with introduced cyclic dithio group was isolated using HPLC and identified by MALDI-TOF.

### Example 24

### Drug-BPB Conjugates

### 1. Preparation and Identification of Docetaxel(DTX)-NHS

The hydroxyl group of DTX was reacted with succinic anhydride for 12 hr at room temperature to give DTX succinic acid. For conjugation of the free amine group of lysine residue of BPB with DTX succinic acid, a NHS group reactable with amine was linked to DTX succinic acid. DTX succinic acid was dissolved in MC (methylene choloride) solution, to which EDC/NHS was added and reacted for 12 hr at room temperature. At this time, the mole ratio of DTX succinic acid : EDC : NHS is 1:1.2:1.5. After the reaction, the reaction product was identified by TLC (thin liquid chromatography) using EA (ethyl acetate) as mobile phase solvent.

2. Conjugation of DTX-NHS with BPB

DTX-NHS was dissolved in DMF immediately before conjugation and then BPB was added. If completely dissolved, a small amount of DMSO was additionaly added. To the resultant, DIEA (di-isoethyl amine) was added to promote formation of free amine of BPB and reacted for 12 hr at room temperature. Two equivalents of DIEA to BPB were used. The BPB used was N-terminal acetylated BPB and its sequence was SSSPIQGSWTWENGKWTWRGIIRLEQ.

### 3. Purification and Analysis of DTX-BPB

The conjugation resultant of DTX-NHS and BPB was dried in vaccum dryer and dissolved in 50% ACN, followed by HPLC analysis. The HPLC peak sample corresponding to the DTX-BPB conjugate was analyzed by MALD TOF to verify the formation of the DTX-BPB conjugate.

Having described a preferred embodiment of the present invention, it is to be understood that variants and modifications thereof falling within the spirit of the invention may become apparent to those skilled in this art, and the scope of this invention is to be determined by appended claims and their equivalents.

What is claimed is:

1. A BPB-based cargo delivery system, comprising:
(a) a bipodal peptide binder (BPB) comprising:
(i) a structure stabilizing region comprising a parallel amino acid strand, an antiparallel amino acid strand or a parallel and an antiparallel amino acid strands to induce interstrand non-covalent bonds; and
(ii) a target binding region I and a target binding region II each binding to each of both termini of the structure stabilizing region, wherein the number of amino acid residues of the target binding region I is n and the number of amino acid residues of the target binding region II is m; and
(b) a cargo linked to the bipodal peptide binder.

2. The delivery system according to claim 1, wherein the interstrand non-covalent bonds comprise a hydrogen bond, an electrostatic interaction, a hydrophobic interaction, a Van der Waals interaction, a pi-pi interaction, a cation-pi interaction or a combination thereof.

3. The delivery system according to claim 1, wherein the amino acid strands in the structure stabilizing region are linked by a linker.

4. The delivery system according to claim 1, wherein the structure stabilizing region is a β-hairpin motif, a β-turn motif, a β-sheet motif linked by a linker, a leucine-zipper motif, a leucine-zipper motif linked by a linker, a leucine-rich motif or a leucine-rich motif linked by linker.

5. The delivery system according to claim 1, wherein the structure stabilizing region is a β-hairpin motif.

6. The delivery system according to claim 5, wherein the β-hairpin motif comprises the amino acid sequence selected from the group consisting of SEQ ID NOs: 1-19.

7. The delivery system according to claim 6, wherein the β-hairpin motif comprises the amino acid sequence selected from the group consisting of SEQ ID NO: 2, SEQ ID NO: 14 or SEQ ID NO: 18.

8. The delivery system according to claim 5, wherein the β-hairpin motif is represented by the following Formula I:
X₁-Trp(X₂)X₃-X₄-X₅(X′₂)X₆-X₇ Formula I
wherein X₁ represents Ser or Gly-Glu, and X₂ and X′₂ independently represent Thr, His, Val, Ile, Phe or Tyr, and X₃ represents Trp or Tyr, and X₄ represents type I, type I′, type II, type II′, type III or type III′ turn sequence, and X₅ represents Trp or Phe, and X₆ represents Trp or Val, and X₇ represents Lys or Thr-Glu.

9. The delivery system according to claim 5, wherein the β-hairpin motif is represented by the following Formula II:
X₁-Trp-X₂-Tyr-X₃-Phe-Thr-Val-X₄ Formula II
wherein X₁ represents Arg, Gly-Glu or Lys-Lys, and X₂ represents Gln or Thr, and X₃ represents type I, type I′, type II, type II′, type III or type III′ turn sequence, and X₄ represents Gln, Thr-Glu or Gln-Glu.

10. The delivery system according to claim 5, wherein the β-hairpin motif is represented by the following Formula III:
X₁-X₂-X₃-Trp-X₄-X₅-Thr-X₆-X₇ Formula III
wherein X₁ represents Lys or Lys-Lys, and X₂ represents Trp or Tyr, and X₃ represents Val or Thr, and X₄ represents type I, type I′, type II, type II′, type III or type III′ turn sequence, and X₅ represents Trp or Ala, and X₆ represents Trp or Val, and X₇ represents Glu or Gln-Glu.

11. The delivery system according to claim 5, wherein the β-hairpin motif is represented by the following Formula IV:
X₁-X₂-X₃-Trp-X₄ Formula IV
wherein X₁ represents Lys-Thr or Gly, and X₂ represents Trp or Tyr, and X₃ represents type I, type I′, type II, type II′, type III or type III′ turn sequence, and X₄ represents Thr-Glu or Gly.

12. The delivery system according to claim 8, wherein the β-hairpin motif is represented by the Formula I in which X₁ represents Ser or Gly-Glu, and X₂ and X′₂ independently represent Thr, His or Val, and X₃ represents Trp or Tyr, and X₄ represents type I, type I′, type II or type II′ turn sequence, and X₅ represents Trp or Phe, and X₆ represents Trp or Val, and X₇ represents Lys or Thr-Glu.

13. The delivery system according to claim 1, wherein the n in the target binding region I is in a range of 2-20.

14. The delivery system according to claim 1, wherein the m in the target binding region I is in a range of 2-20.

15. The delivery system according to claim 1, wherein the target binding region I and the target binding region II bind in a cooperative manner to the target.

16. The delivery system according to claim 1, wherein the structure stabilizing region, the target binding region I or the target binding region II further comprises a functional molecule.

17. The delivery system according to claim 16, wherein the functional molecule comprises a cell penetrating peptide (CPP).

18. The delivery system according to claim 1, wherein the target comprises a biochemical material, a peptide, a polypeptide, a nucleic acid, a carbohydrate, a lipid, a cell, a tissue, a compound, a metal material or a non-metal material.

19. The delivery system according to claim 1, wherein the cargo comprises a chemical compound, a chemical drug, a biodrug, an inorganic particle, a nanoparticle, a protein, a peptide, a nucleic acid molecule, a lipid, a carbohydrate, a liposome or a label capable of generating a detectable signal.


SEQUENCE LISTING (41 sequences)

SEQ ID NO: 1 - protein, 12 aa, Artificial Sequence
  Trpzip1
SWTWEGNKWTWK

SEQ ID NO: 2 - protein, 12 aa, Artificial Sequence
  Trpzip2
SWTWENGKWTWK

SEQ ID NO: 3 - protein, 12 aa, Artificial Sequence
  Trpzip3
SWTWEPNKWTWK

SEQ ID NO: 4 - protein, 16 aa, Artificial Sequence
  gb1, 41-56
GEWTYDDATKTFTVTE

SEQ ID NO: 5 - protein, 16 aa, Artificial Sequence
  Trpzip4
GEWTWDDATKTWTWTE

SEQ ID NO: 6 - protein, 16 aa, Artificial Sequence
  Trpzip5
GEWTYDDATKTFTWTE

SEQ ID NO: 7 - protein, 16 aa, Artificial Sequence
  Trpzip6
GEWTWDDATKTWTVTE

SEQ ID NO: 8 - protein, 16 aa, Artificial Sequence
  Trpzip7
GEWHWDDATKTWVWTE

SEQ ID NO: 9 - protein, 16 aa, Artificial Sequence
  Trpzip8
GEWVWDDATKTWHWTE

SEQ ID NO: 10 - protein, 16 aa, Artificial Sequence
  Trpzip9
GEWVWDDATKTWVWTE

SEQ ID NO: 11 - protein, 12 aa, Artificial Sequence
  (T3V)-HP6
KYVWSNGKWTVE

SEQ ID NO: 12 - protein, 12 aa, Artificial Sequence
  Espinosa-GB1(b)
RWQYVNGKFTVQ

SEQ ID NO: 13 - protein, 16 aa, Artificial Sequence
  GB1m2
GEWTYNPATGKFTVTE

SEQ ID NO: 14 - protein, 16 aa, Artificial Sequence
  GB1m3
KKWTYNPATGKFTVQE

SEQ ID NO: 15 - protein, 16 aa, Artificial Sequence
  HP5W4
KKWTYNPATGKWTWQE

SEQ ID NO: 16 - protein, 16 aa, Artificial Sequence
  HP5W
KKYTWNPATGKWTVQE

SEQ ID NO: 17 - protein, 16 aa, Artificial Sequence
  HP5A
KKYTWNPATGKATVQE

SEQ ID NO: 18 - protein, 12 aa, Artificial Sequence
  HP7
KTWNPATGKWTE

SEQ ID NO: 19 - protein, 10 aa, Artificial Sequence
  Chignolin
GYDPETGTWG

SEQ ID NO: 20 - protein, 26 aa, Artificial Sequence
  BPB-EB-B1
MSADKSGSWTWENGKWTWKGQVRTRD

SEQ ID NO: 21 - protein, 26 aa, Artificial Sequence
  BPB-ED-B2
HCSSAVGSWTWENGKWTWKGIIRLEQ

SEQ ID NO: 22 - protein, 26 aa, Artificial Sequence
  BPB-ED-B3
HSQGSPGSWTWENGKWTWKGRYSHRA

SEQ ID NO: 23 - protein, 26 aa, Artificial Sequence
  BPB-VEGF1
HANFFQGSWTWENGKWTWKGWKYNQS

SEQ ID NO: 24 - protein, 26 aa, Artificial Sequence
  BPB-VEGF2
ASPFWAGSWTWENGKWTWKGWVPSNA

SEQ ID NO: 25 - protein, 26 aa, Artificial Sequence
  BPB-VEGF3
HAFYYTGSWTWENGKWTWKGWPVTTS

SEQ ID NO: 26 - protein, 26 aa, Artificial Sequence
  BPB-VEGF4
YGAYPWGSWTWENGKWTWKGWRVSRD

SEQ ID NO: 27 - protein, 26 aa, Artificial Sequence
  BPB-VEGF5
AAPTSFGSWTWENGKWTWKGWQMWHR

SEQ ID NO: 28 - protein, 26 aa, Artificial Sequence
  BPB-VCAM1
QARDCTGSWTWENGKWTWKGPSICPI

SEQ ID NO: 29 - protein, 26 aa, Artificial Sequence
  BPB-nAchR1
EASFWLGSWTWENGKWTWKGKGTLNR

SEQ ID NO: 30 - protein, 26 aa, Artificial Sequence
  BPB-nAchR2
YAYPLLGSWTWENGKWTWKGWYQKWI

SEQ ID NO: 31 - protein, 26 aa, Artificial Sequence
  BPB-nAchR3
ASLPAWGSWTWENGKWTWKGWSTRTA

SEQ ID NO: 32 - protein, 26 aa, Artificial Sequence
  BPB-BSA1
AASPYKGSWTWENGKWTWKGGWRMKM

SEQ ID NO: 33 - protein, 26 aa, Artificial Sequence
  BPB-BSA2
SANSLYGSWTWENGKWTWKGTSRQRW

SEQ ID NO: 34 - protein, 26 aa, Artificial Sequence
  BPB-BSA3
YAHVYYGSWTWENGKWTWKGHRVTQT

SEQ ID NO: 35 - protein, 26 aa, Artificial Sequence
  BPB-BSA4
YGAYPWGSWTWENGKWTWKGWRVSRD

SEQ ID NO: 36 - protein, 26 aa, Artificial Sequence
  BPB-BSA5
YAHFGWGSWTWENGKWTWKGTTDSQS

SEQ ID NO: 37 - protein, 26 aa, Artificial Sequence
  BPB-MyD88-1
HSHAFYGSWTWENGKWTWKGNPGWWT

SEQ ID NO: 38 - protein, 26 aa, Artificial Sequence
  BPB-MyD88-2
ASTINFGSWTWENGKWTWKGYTRRWN

SEQ ID NO: 39 - protein, 35 aa, Artificial Sequence
  Leucine zipper for BPB
GGSMKQLEDKVEELLSKNYHLENEVARLKKLVGER

SEQ ID NO: 40 - protein, 41 aa, Artificial Sequence
  BPBLeu-ED-B-1
CSSPIQGGSMKQLEDKVEELLSKNYHLENEVARLKKLVGER

SEQ ID NO: 41 - protein, 41 aa, Artificial Sequence
  BPBLeu-ED-B-2
IIRLEQGGSMKQLEDKVEELLSKNYHLENEVARLKKLVGER